(19) United States
(12) Patent Application Publication
(10) Pub. No.: US 2025/0163481 A1
(43) Pub. Date: May 22, 2025

(54) IMPROVED BIOTECHNOLOGICAL METHOD FOR PRODUCING GUANIDINO ACETIC ACID (GAA) BY USING NADH-DEPENDENT DEHYDROGENASES

(71) Applicant: Evonik Operations GmbH, Essen (DE)

(72) Inventors: Frank Schneider, Halle (DE); Steffen Schaffer, Herten (DE); Kay Marin, Borgholzhausen (DE); Melanie Nickolaus, Spenge (DE); Julia Tegethoff, Halle (Westf.) (DE); Marleen Oesterhoff, Paderborn (DE)

(73) Assignee: Evonik Operations GmbH, Essen (DE)

(21) Appl. No.: 18/869,988

(22) PCT Filed: May 24, 2023
(86) PCT No.: PCT/EP2023/063897

§ 371 Date: Nov. 27, 2024

(30) Foreign Application Priority Data
Jun. 3, 2022 (EP) 22177262.7

(51) Int. Cl. C12P 13/04; C12N 9/00; C12N 9/06; C12N 9/10; C12N 9/88; C12N 15/77

(52) U.S. Cl. CPC C12P 13/04; C12N 9/0016; C12N 9/1003; C12N 9/1018; C12N 9/88; C12N 9/93; C12N 15/77; C12Y 104/01001; C12Y 104/0101; C12Y 104/01021; C12Y 201/01002; C12Y 201/03003; C12Y 201/04001; C12Y 403/02001; C12Y 603/04005; C12Y 603/04016

(57) ABSTRACT

A microorganism produces guanidinoacetic acid (GAA) and has at least one gene coding for a protein having the function of a NADH-dependent dehydrogenase. A method for the fermentative production of GAA uses such microorganism. A method produces creatine through fermentative production. Industrial feed stocks are used as starting material in the fermentative process.

Guanidino acetic acid (GAA) is a colourless crystalline organic compound used as animal feed additive (e.g. WO 2005120246 A1 and US 2011257075 A1). GAA is a natural precursor of creatine (e.g. Humm et al., Biochem. J. (1997) 322, 771-776). Therefore, the supplementation of GAA allows for an optimal supply of creatine in the organism.

The present invention pertains to a microorganism transformed to be capable of producing guanidinoacetic acid (GAA) and to a method for the fermentative production of GAA using such microorganism. The present invention also relates to a method for the fermentative production of creatine. For the fermentative process industrial feed stocks (e.g. ammonia, ammonium salts and glucose or sugar containing substrates) are used as starting material.

In biological systems GAA and omithine are formed from arginine and glycine as starting materials by the catalytic action of an L-arginine:glycine-amidinotransferase (AGAT; EC 2.1.4.1). This reaction is also the first step in creatine biosynthesis.

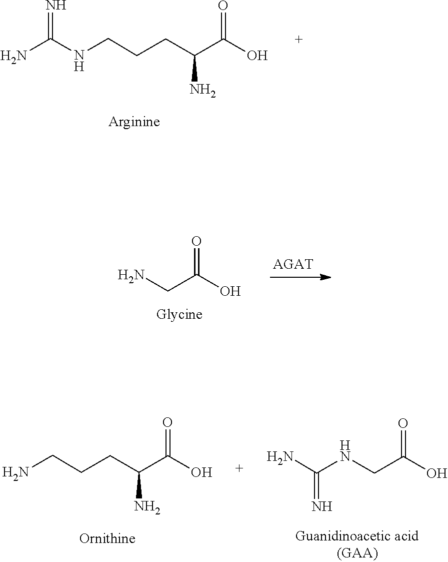 [SMILES: N=C(N)NCCC[C@H](N)C(=O)O ; NCC(=O)O ; NCCC[C@H](N)C(=O)O ; N=C(N)NCC(=O)O]

Guthmiller et al. (J Biol Chem. 1994 Jul. 1; 269(26):17556-60) have characterized a rat kidney AGAT by cloning and heterologously expressing the enzyme in E. coli. Muenchhoff et al. (FEBS Journal 277 (2010) 3844-3860) report the first characterization of an AGAT from a prokaryote also by cloning and heterologously expressing the enzyme in E. coli. Sosio et al. (Cell Chemical Biology 25, 540-549, May 17, 2018) elucidated the biosynthetic pathway for 25 pseudouridimycin in Streptomyces sp. They describe as an intermediate reaction the formation of GAA and L-omithine by the reaction of L-arginine with glycine catalyzed by PumN, an Larginine:glycine-amidinotransferase (AGAT).

Fan Wenchao discloses a method for the production of creatine by fermentation of non-pathogenic microorganisms, such as Corynebacterium glutamicum (CN 106065411 A). The microorganism has the following biotransformation functions: glucose conversion to L-glutamic acid; conversion of L-glutamic acid to N-acetyl-L-glutamic acid; conversion of N-acetyl-L-glutamic acid to N-acetyl-L-glutamic acid semialdehyde; conversion of N-acetyl-L-glutamic acid semialdehyde to N-acetyl-L-omithine; conversion of N-acetyl-L-omithine to L-omithine; conversion of L-omithine to L-citrulline; conversion of L-citrulline to arginino-succinic acid; conversion of arginino-succinic acid to L-arginine; conversion of L-arginine to guanidinoacetic acid; and, finally, conversion of guanidinoacetic acid to creatine. Fan Wenchao proposes, that the microorganism overexpresses one or more enzymes selected from the group consisting of N-acetylglutamate-synthase, N-acetylornithine-δ-aminotransferase, N-acetylornithinase, omithine-carbamoyl transferase, argininosuccinate synthetase, glycine amidino-transferase (EC: 2.1.4.1), and guanidinoacetate N-methyltransferase (EC: 2.1.1.2). The microorganism overexpresses preferably glycine aminotransferase (L-arginine:glycine amidinotransferase) and guanidinoacetate N-methyltransferase.

CN 113481139 A describes the construction of a recombinant Bacillus subtilis producing GAA by introducing exogenous arginine:glycine amidinotransferase from Amycolatopsis kentuckyensis into the genome of a wild-type Bacillus subtilis and by knocking out the gcvP and/or argI gene(s) in the genome.

A microorganism capable of producing guanidinoacetic acid (GAA) was published by Zhang et al. (ACS Synth. Biol. 2020, 9, 2066-275). They designed a reconstituted omithine cycle in Escherichia coli by introducing a heterologous AGAT from different species (e.g., Homo sapiens, Cylindrospemopsis raciborskii, Moorea producens) and by introducing a citrulline synthesis module (e.g. coexpression of carAB, argF and arg) and an arginine synthesis module (e.g. overexpression of argG, argH; introduction of aspA) into Escherichia coli.

Several approaches for increasing the production of one of the starting materials in GAA synthesis, i.e. L-arginine, in microorganisms, particularly bacteria, are also known from literature. An overview for the metabolic engineering of Corynebacterium glutamicum (C. glutamicum) for L-arginine production is provided by Park et al. (NATURE COMMUNICATIONS|DOI: 10.1038/ncomms5618). They propose random mutagenesis and screening for L-arginine producers of already L-arginine producing C. glutamicum strains, e.g. of ATCC 21831 (Nakayama and Yoshida 1974, U.S. Pat. No. 3,849,250 A) and stepwise rational metabolic engineering based on system-wide analysis of metabolism results in a gradual increase in L-arginine production throughout the strain engineering steps. Yim et al. (J Ind Microbiol Biotechnol (2011) 38:1911-1920) could show that inactivation of the argR, gene coding for the central repressor protein ArgR controlling the L-arginine biosynthetic pathway, by disrupting the chromosomal argR gene in C. glutamicum leads to an improved arginine-producing strain. Ginesy et al. (Microbial Cell Factories (2015) 14:29) report the successful engineering of E. coli for enhanced arginine production. Among other, they proposed the deletion of the argR repressor gene.

Kurahashi et al. (EP 1057893 A1) report methods for increasing the L-arginine producing ability of a microorganism by enhancing L-arginine biosynthesis enzymes utilizing recombinant DNA techniques, e.g. by utilizing a microorganism belonging to the genus Corynebacterium or Brevibacterium which is made to harbor a recombinant DNA comprising a vector DNA and a DNA fragment containing genes for acetylornithine deacetylase, N-acetylglutamic acid-γ-semialdehyde dehydrogenase, N-acetyl glutamokinase and argininosuccinase derived from a microorganism belonging to the genus Escherichia. For an improved L-arginine production the authors further propose a microorganism which is enhanced in an activity of intracellular glutamate dehydrogenase (GDH) and which has an L-arginine producing ability.

Finally, Schneider and Jankowitsch (WO 2021122400 A1; EP 3839051 A1) propose a method for the fermentative production of GAA by using microorganisms having a heterologous AGAT from different species (e.g., Homo sapiens, Cylindrospermopsis raciboskii, Moorea producens) and having an improved ability to produce L-arginine, e.g. by increased expression of enzymes of the L-arginine pathway, such as of an enzyme having the function of a carbamoylphosphate synthase and of enzymes related to the arginine operon.

As to the second starting material of the GAA biosynthesis, it would be also desirable increasing the provision of glycine in order to improve the GAA biosynthesis in microorganisms that are naturally provided with a homologous gene coding for a protein having the function of a L-arginine:glycine amidinotransferase (AGAT) or have been provided with a heterologous gene coding for a protein having the function of a L-arginine:glycine amidinotransferase (AGAT).

Schneider and Jankowitsch (WO 2022008280 A1) disclose a method to produce GAA by using a microorganism comprising at least one gene coding for a protein having the function of a L-arginine:glycine amidinotransferase (AGAT) and at least one protein having the function of a glyoxylate aminotransferase. By using a glyoxylate aminotransferase it was shown that the yield of GAA was increased.

Several glyoxylate amino transferases are known and vary in their substrate specificity with respect to the amino donor (cf. e.g. Kameya et al. FEBS Journal 277 (2010) 1876-1885; Liepman and Olsen, Plant Physiol. Vol. 131, 2003, 215-227; Sakuraba et al., JOURNAL OF BACTERIOLOGY, August 2004, p. 5513-5518; Takada and Noguchi, Biochem. J. (1985) 231, 157-163). Since most of these glyoxylate amino transferase are able to use different amino acids as amino donors, they are often annotated with different EC numbers. However, all these aminotransferases have in common that they use glyoxylate as acceptor molecule, or, in case of the reverse reaction, glycine as donor molecule. It could also be shown that the decrease of the activity of a protein having the function of a malate synthase that uses glyoxylate as substrate in a microorganism provided with a gene coding for a protein having the function of an AGAT leads to an increase of GAA yield (WO 2022008276 A1).

The disadvantage of glyoxylate amino transferases is that the amino group of the amino donor is normally derived from L-glutamate or L-glutamate is used directly. The biosynthesis of L-glutamate from 2-oxoglutarate in general requires NADPH. In C. glutamicum NADPH is regenerated from NADP+ mainly through an oxidative pentose phosphate pathway (PPP) and partly by NADP-dependent isocitrate dehydrogenase and NADP-dependent malic enzyme (Marx, Achim, de Graaf, Albert A., Wiechert, Wolfgang, Lothar Eggeling and Sahm, Hermann (1996), Biotechnology and Bioengineering, Vol 49(2), 111-129, DOI: https://doi.org/10.1002/(SICI)1097-0290(19960120)49:2<111::AID-BIT1>3.0.CO;2-T). These pathways for NADPH-regeneration result in carbon loss and thereby lower the maximal theoretical yield. On the other hand, methods of enhancing the NADPH supply are known, e.g.: overexpression of inorganic polyphosphate/ATP-NAD kinase ppnK (Yin, Lianghong; Zhao, Jianxun; Chen, Cheng; Hu, Xiaoqing; Wang, Xiaoyuan. (2014) Biotechnology and Bioprocess Engineering: BBE; Dordrecht Bd. 19, Ed. 1: 132-142. DOI:10.1007/s12257-013-0416-z) or the expression of a heterologous transhydrogenase gene (Yamauchi Y, Hirasawa T, Nishii M, Furusawa C, Shimizu H. (2014) J Gen Appl Microbiol. 2014; 60(3):112-8. doi: 10.2323/jgam.60.112. PMID: 25008167). C. glutamicum naturally fixes NH₃ with NADPH-dependent glutamate dehydrogenase (E. Kimura (2005) “L-Glutamate Production” in Handbook of Corynebacterium glutamicum, ISBN 9780849318214, Published Mar. 30, 2005 by CRC Press). It is known that this is also possible with heterologous NADH-dependent glutamate dehydrogenase (Marx, Achim, Eikmanns, Bernhard J., Sahm, Hermann, de Graaf, Albert A. and Lothar Eggeling (1999) Metabolic Engineering, Volume 1, Issue 1, 1999, Pages 35-48, ISSN 1096-7176, DOI: https://doi.org/10.1006/mben.1998.0106). This reduces the NADPH requirement.

NADH depending amino acid dehydrogenases (AaDH) catalyse the amination reaction of a keto acid to a L-amino acid by utilizing NADH as a cosubstrate (instead of NADPH). They therefore provide an alternative pathway for the assimilation or dissimilation of ammonium in a cell.

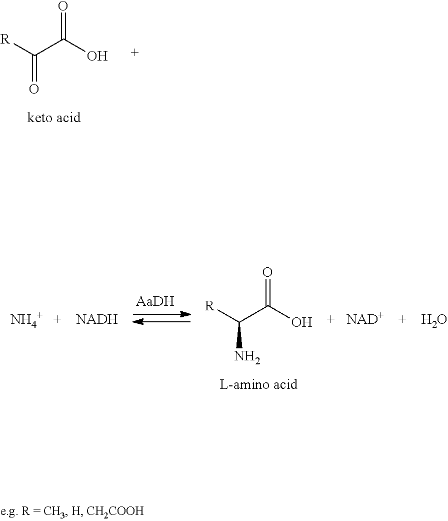

Since most of these amino acid dehydrogenases are able to use different a-keto acids as substrate, they are often annotated with different EC numbers. However, all these amino acid dehydrogenases have in common that they assimilate ammonium, or, in case of the reverse reaction, dissimilate ammonium.

Examples for different amino acid dehydrogenases are the following:

### Reaction EC 1.4.1.1: Alanine Dehydrogenase:

pyruvate+NH3+NADH+H+<->L-alanine+H2O+NAD+

### Reaction EC 1.4.1.10: Glycine Dehydrogenase:

glyoxylate+NH3+NADH+H+<->gycine+H2O+NAD+

### Reaction EC 1.4.1.21: Aspartate Dehydrogenase:

oxaloacetate+NH3+NADH+H+<->L-aspartate+H2O+NAD+

Several NADH dependent amino acid dehydrogenase (AaDH) Proteins are known in literature which accept a broad range of substrates and can therefore often aminate several different keto acids to the corresponding amino acids. (Feandes et al. Protein Engineering, Design & Selection, 2015, vol. 28 no. 2, pp. 29-35; Giffin et al., Journal of Bacteriology, 2012, vol. 194 no. 5, pp. 1045-1054; Phogosee et al., Archives of Microbiology, 2018 vol. 200 pp. 719-727; Schuffenhauer et al. 1999, vol. 171, pp 417-423; Vancura et al. Eur J Biochem 1989, vol. 179, pp. 221-227, Yoshida and Freese, Biochim. Biophys. Acta, 1965, vol. 96, pp 248-262)

Therefore, most NADH depending amino acid dehydrogenases correspond to multiple reactions and thus multiple EC numbers. Table 1 shows some examples:

TABLE 1
Examples for NADH depending amino acid dehydrogenase (AaDH) Proteins

| | AaDH | AaDH | AaDH | AaDH | AaDH |
|---|---|---|---|---|---|
| | Mycobacterium | Mycobacterium | Bacillus | Streptomyces | Aphanothece |
| Reaction | tuberculosis | smegmatis | subtilis | fradiae | halophytica |
| Pyruvat->L-Alanin | yes (1) | yes (3) | yes (4) | yes (5) | yes (6) |
| L-Alanin->Pyruvat | yes (1) | yes (3) | yes (4) | yes (5) | yes (6) |
| Glyoxylat->Glycin | yes (1) | yes (3) | yes (4) | no(5) | yes (6) |
| Oxalacetat->Aspartat | yes (1) | yes (3) | no data | yes (5) | no data |
| Hydroxypyruvat -> | yes (1) | no data | yes (4) | no data | no data |
| Serin | | | | | |
| Methylglyoxal -> | yes (1) | no data | no data | no data | no data |
| Aminoaceton | | | | | |
| 4-Hydroxy-2- | yes (2) | no data | no data | no data | no data |
| oxobutyrat -> L- | | | | | |
| Homoserin | | | | | |
| 2-Oxobutyrate -> 2- | no data | yes (3) | yes (4) | yes(5) | no data |
| aminobutyrate | | | | | |
| Literature | (1) Giffin, 2012 | (3) | (4) | (5) | (6) |
| | (2) Fernandes, | Schuffen- | Yoshida, | Vancura, | Phogosee, |
| | 2015 | hauer, | 1965 | 1989 | 2019 |
| | | 1999 | | | |

Therefore, the problem underlying the present invention is to provide an improved microorganism transformed to be capable of producing guanidinoacetic acid (GAA), in particular a microorganism with an improved capacity of providing glycine as starting material of the GAA biosynthesis, and to a method for the fermentative production of GAA using such microorganism.

The problem is solved by a microorganism comprising at least one heterologous gene coding for a protein having the function of a L-arginine:glycine amidinotransferase (AGAT, e.g. EC 2.1.4.1) and comprising at least one heterologous gene coding for a protein having the function of a NADH-dependent amino acid dehydrogenase.

A heterologous gene means that the gene has been inserted into a host organism which does not naturally have this gene. Insertion of the heterologous gene in the host is performed by recombinant DNA technology. Microorganisms that have undergone recombinant DNA technology are called transgenic, genetically modified or recombinant.

A heterologous protein means a protein that is not naturally occurring in the microorganism.

A homologous or endogenous gene means that the gene including its function as such or the nucleotide sequence of the gene is naturally occurring in the microorganism or is “native” in the microorganism.

A homologous or a native protein means a protein that is naturally occurring in the microorganism.

Proteins having the function of an L-arginine:glycine amidinotransferase (AGAT) belong to the amidinotransferase family. The amidinotransferase family comprises glycine (EC:2.1.4.1) and inosamine (EC:2.1.4.2) amidinotransferases, enzymes involved in creatine and streptomycin biosynthesis respectively. This family also includes arginine deiminases, EC:3.5.3.6. These enzymes catalyse the reaction: arginine+H2O<=>citrulline+NH3. Also found in this family is the Streptococcus anti tumour glycoprotein. Enzymes or proteins with an L-arginine:glycine-amidinotransferase (AGAT) activity are also described to possess a conserved domain that belongs to the PFAM Family: Amidinotransf (PF02274) (Marchler-Bauer A et al. (2017), “CDD/SPARCLE: functional classification of proteins via subfamily domain architectures.”, Nucleic Acids Res. 45(D1):D200-D203.) as described also in the following publications: Pissowotzki K et al., Mol Gen Genet 1991; 231:113-123 (PUBMED:1661369 EPMC:1661369); D'Hooghe I et al., J Bacteriol 1997; 179:7403-7409 (PUBMED:9393705 EPMC:9393705); Kanaoka M et al., Jpn J Cancer Res 1987; 78:1409-1414 (PUBMED:3123442 EPMC:3123442).

In the microorganism according to the present invention the at least one protein having the function of a NADH-dependent amino acid dehydrogenase may be a NADH depending amino acid dehydrogenase. It may be selected from the group consisting of proteins having the function of an alanine dehydrogenase (EC 1.4.1.1), of a glycine dehydrogenase (EC 1.4.1.10) and of an aspartate dehydrogenase (EC 1.4.1.21).

In a further embodiment of the microorganism of the present invention the activity of the NADH-dependent amino acid dehydrogenase is increased compared with the respective activity in the wildtype microorganism. Preferably, at least one gene encoding the protein having the enzymic activity of a NADH-dependent amino acid dehydrogenase is overexpressed in the microorganism of the present invention compared to the expression of the respective gene in the wildtype microorganism.

Preferably, the microorganism of the present invention has an increased ability to produce L-arginine from L-omithine compared with the ability of the wildtype microorganism.

In the context of the present invention, a microorganism having an increased ability to produce L-arginine means a microorganism producing L-arginine in excess of its own need. Examples for such L-arginine producing microorganisms are e.g. C. glutamicum ATCC 21831 or those disclosed by Park et al. (NATURE COMMUNICATIONS|DOI: 10.1038/ncomms5618) or by Ginesy et al. (Microbial Cell Factories (2015) 14:29).

In a particular embodiment of the present invention, the microorganism has an increased activity of an enzyme having the function of a carbamoylphosphate synthase (EC 6.3.4.16) compared to the respective enzymatic activity in the wildtype microorganism.

The activity of an enzyme having the function of an argininosuccinate lyase (E.C. 4.3.2.1) in the microorganism according to the present invention may be increased compared to the respective enzymic activity in the wildtype microorganism.

Furthermore, in the microorganism according to the present invention, the activity of an enzyme having the function of an omithine carbamoyltransferase (EC 2.1.3.3) may be increased compared to the respective enzymic activity in the wildtype microorganism.

In the microorganism according to the present invention, the activity of an enzyme having the function of an argininosuccinate synthetase (E.C. 6.3.4.5) may be also increased compared to the respective enzymic activity in the wildtype microorganism.

In a further embodiment of the microorganism according to the present invention the expression of a gene encoding the protein having the function of a malate synthase is attenuated compared to the expression of the respective gene in the wildtype microorganism or a gene encoding the protein having the function of a malate synthase is inactivated or deleted.

In a further embodiment the microorganism according to the present invention the expression of an argR gene coding for the arginine responsive repressor protein ArgR is attenuated compared to the expression of the argR gene in the wildtype microorganism. Alternatively, the argR gene is inactivated or deleted.

In a further embodiment of the present invention and, optionally in addition to the above-mentioned modifications, at least one or more of the genes coding for an enzyme of the biosynthetic pathway of L-arginine, comprising of gdh, argJ, argB, argC and/or argD coding for a glutamate dehydrogenase, an omithine acetyltransferase, an acetylglutamate kinase, an acetylglutamylphosphate reductase and an acetylornithine aminotransferase, respectively, is overexpressed in the microorganism according to the present invention.

In the microorganism according to the present invention the protein having the function of a L-arginine:glycine amidinotransferase may be a L-arginine:glycine amidinotransferase (AGAT, i.e. EC 2.1.4.1).

In the microorganism of the present invention the gene coding for a protein having the function of an L-arginine:glycine amidinotransferase may further be overexpressed.

The protein having the function of an L-arginine:glycine amidinotransferase (AGAT) in the microorganism of the present invention may comprise an amino acid sequence which is at least 80% identical, preferably at least 90% identical to the amino acid sequence according to SEQ ID NO: 2. In a further embodiment of the present invention the amino acid sequence of the L-arginine:glycine amidinotransferase is identical to amino acid sequence according to SEQ ID NO: 2.

In a particular embodiment of the present invention, the protein having the function of a NADH-dependent amino acid dehydrogenase comprises an amino acid sequence which is at least 80% identical to the amino acid sequence according to SEQ ID NO: 6, according to SEQ ID NO: 9, according to SEQ ID NO: 12, according to SEQ ID NO: 15 or according to SEQ ID NO: 18.

The microorganism of the present invention may belong to the genus Corynebacterium, preferably Corynebacterium glutamicum (C. glutamicum), or to the genus Enterobacteriaceae, preferably Escherichia coli (E. coli), or to the genus Pseudomonas, preferably Pseudomonas putida (P. putida).

Generally, an increased enzymic activity of a protein in a microorganism, in particular in the microorganism of the present invention compared to the respective activity in the wildtype microorganism, can be achieved for example by a mutation of the protein, in particular by a mutation conferring the protein a feedback resistance e.g. against a product of an enzyme-catalyzed reaction, or by overexpression of a gene encoding the protein having the enzymic activity compared to the expression of the respective gene in the wildtype microorganism.

Overexpression of a gene is generally achieved by increasing the copy number of the gene and/or by functionally linking the gene with a strong promoter and/or by enhancing the ribosomal binding site and/or by codon usage optimization of the start codon or of the whole gene or a combination comprising a selection of all methods mentioned above.

Overexpression of a gene in a microorganism, in particular in the microorganism of the present invention compared to the respective activity in the wildtype microorganism, can be achieved by increasing the copy number of the gene and/or by an enhancement of regulatory factors, e.g. by functionally linking the gene with a strong promoter and/or by enhancing the ribosomal binding site and/or by codon usage optimization of the start codon or of the whole gene. The enhancement of such regulatory factors which positively influence gene expression can, for example, be achieved by modifying the promoter sequence upstream of the structural gene in order to increase the effectiveness of the promoter or by completely replacing said promoter with a more effective or a so-called strong promoter. Promoters are located upstream of the gene. A promoter is a DNA sequence consisting of about 40 to 50 base pairs and which constitutes the binding site for an RNA polymerase holoenzyme and the transcriptional start point, whereby the strength of expression of the controlled polynucleotide or gene can be influenced. Generally, it is possible to achieve an overexpression or an increase in the expression of genes in bacteria by selecting strong promoters, for example by replacing the original promoter with strong, native (originally assigned to other genes) promoters or by modifying certain regions of a given, native promoter (for example its so-called −10 and −35 regions) towards a consensus sequence, e.g. as taught by M. Patek et al. (Microbial Biotechnology 6 (2013), 103-117) for C. glutamicum. An example for a “strong” promoter is the superoxide dismutase (sod) promoter (“Psod”; Z. Wang et al., Eng. Life Sci. 2015, 15, 73-82). A “functional linkage” is understood to mean the sequential arrangement of a promoter with a gene, which leads to a transcription of the gene.

The genetic code is degenerated which means that a certain amino acid may be encoded by a number of different triplets. The term codon usage refers to the observation that a certain organism will typically not use every possible codon for a certain amino acid with the same frequency.

Instead, an organism will typically show certain preferences for specific codons meaning that these codons are found more frequently in the coding sequence of transcribed genes of an organism. If a certain gene foreign to its future host, i.e. from a different species, should be expressed in the future host organism the coding sequence of said gene should then be adjusted to the codon usage of said future host organism (i.e. codon usage optimization).

The above-mentioned problem is further solved by a method for the fermentative production of guanidino acetic acid (GAA), comprising the steps of a) cultivating the microorganism according to the present invention as defined above in a suitable medium under suitable conditions, and b) accumulating GAA in the medium to form an GAA containing fermentation broth.

The method of the present invention may further comprise the step of isolating GAA from the fermentation broth.

The present invention further concerns a microorganism as defined above, further comprising a gene coding for an enzyme having the activity of a guanidinoacetate N-methyltransferase (EC: 2.1.1.2). Preferably, the gene coding for an enzyme having the activity of a guanidinoacetate N-methyltransferase is overexpressed.

The present invention also concerns a method for the fermentative production of creatine, comprising the steps of a) cultivating the microorganism according to the present invention comprising a gene coding for an enzyme having the activity of a guanidinoacetate N-methyltransferase in a suitable medium under suitable conditions, and b) accumulating creatine in the medium to form a creatine containing fermentation broth.

Preferably, the method further comprises isolating creatine from the creatine containing fermentation broth. creatine may be extracted from fermentation broth by isoelectric point method and/or ion exchange method. Alternatively, creatine can be further purified by a method of recrystallization in water.

### Sequences:

SEQ ID NO:1: Shows an open reading frame coding for a L-arginine:glycine amidinotransferase from Moorena producens (AGAT, EC 2.1.4.1; locus_tag BJP34_00300).
SEQ ID NO:2: Shows the amino acid sequence derived from SEQ ID NO:1 (Genbank accession Number WP_070390602).
SEQ ID NO:3: Shows the DNA sequence coding for the L-arginine:glycine amidinotransferase from Moorena producens (AGAT, EC 2.1.4.1) optimized for the codon usage of C. glutamicum. The 5′-end of the optimized gene is expanded with a BsaI restriction site, a 5′-UTR sequence for assembly cloning and a ribosomal binding site. At the 3′-end a second stop-codon, a sequence for assembly cloning and a BsaI-site has been added.
SEQ ID NO:4: Shows the E. coli-C. glutamicum shuttle plasmid pLIB_P consisting of the replication origin from pBL1 (for C. glutamicum), the pSC101 replication origin (for E. coli) and a kanamycin resistance gene. Following a unique NotI restriction site it has a strong promoter, two inversely orientated BsaI-sites and the BioBricks Terminator BBa_B1006.
SEQ ID NO:5: Shows the open reading frame MRA_2804 of Mycobacterium tuberculosis H37Ra coding for an NADH dependent amino acid dehydrogenase (Genbank accession CP000611 locus_tag=“MRA_2804”).
SEQ ID NO:6: Shows the amino acid sequence derived from SEQ ID NO: 5 (AaDH-Mt from Mycobacterium tuberculosis H37Ra).
SEQ ID NO:7: Shows the DNA sequence coding for the NADH dependent amino acid dehydrogenase of Mycobacterium tuberculosis H37Ra optimized for the codon usage of C. glutamicum. The DNA sequence is expanded with a 5′-UTR consisting of a BsaI restriction site, a homologous region for assembly cloning, the strong Pg3N3 promoter and a ribosomal binding site. Additionally, the 3′-end is expanded with a random spacer sequence, a homologous region for assembly cloning and a BsaI restriction site.
SEQ ID NO:8: Shows the open reading frame LJ00_13235 of Mycobacterium smegmatis MC2 155 presumably coding for a NADH dependent amino acid dehydrogenase (Genbank accession CP009494 locus_tag=“LJ00_13235).
SEQ ID NO:9: Shows the amino acid sequence derived from SEQ ID NO:8 (AaDH-Ms from Mycobacterium smegmatis MC2 155).
SEQ ID NO:10: Shows the DNA sequence coding for the NADH dependent amino acid dehydrogenase of Mycobacterium smegmatis MC2 155 optimized for the codon usage of C. glutamicum. The DNA sequence is expanded with a 5′-UTR consisting of a BsaI restriction site, a homologous region for assembly cloning, the strong Pg3N3 promoter and a ribosomal binding site. Additionally, the 3′-end is expanded with a random spacer sequence, a homologous region for assembly cloning and a BsaI restriction site.
SEQ ID NO:11: Shows the open reading frame HIR77_18035 of Bacillus subtilis subsp. subtilis str. 168 presumably coding for a NADH dependent amino acid dehydrogenase (Genbank accession CP053102 locus_tag=“HIR77_18035”).
SEQ ID NO:12: Shows the amino acid sequence derived from SEQ ID NO:11 (AaDH-Bs from Bacillus subtilis subsp. subtilis str. 168).
SEQ ID NO:13: Shows the DNA sequence coding for the NADH dependent amino acid dehydrogenase of Bacillus subtilis subsp. subtilis str. 168 optimized for the codon usage of C. glutamicum. The sequence is expanded with a 5′-UTR consisting of a BsaI restriction site, a homologous region for assembly cloning, the strong Pg3N3 promoter and a ribosomal binding site. Additionally, the 3′-end is expanded with a random spacer sequence, a homologous region for assembly cloning and a BsaI restriction site.
SEQ ID NO:14: Shows the open reading frame CP974_05185 of Streptomyces fradiae ATCC10745 presumably coding for a NADH dependent amino acid dehydrogenase (Genbank accession CP023696 locus_tag=“CP974_05185”).
SEQ ID NO:15: Shows the amino acid sequence derived from SEQ ID NO:14 (AaDH-Sf from Streptomyces fradiae ATCC10745).
SEQ ID NO:16: Shows the DNA sequence coding for the NADH dependent amino acid dehydrogenase of Streptomyces fradiae ATCC10745 optimized for the codon usage of C. glutamicum. The sequence is expanded with a 5′-UTR consisting of a BsaI restriction site, a homologous region for assembly cloning, the strong Pg3N3 promoter and a ribosomal binding site. Additionally, the 3′-end is expanded with a random spacer sequence, a homologous region for assembly cloning and a BsaI restriction site.
SEQ ID NO:17: Shows an open reading frame of Aphanothece halophytica CM1 presumably coding for a NADH dependent amino acid dehydrogenase (Genbank accession MG430510).
SEQ ID NO:18: Shows the amino acid sequence derived from SEQ ID NO:17 (AaDH-Ah from Aphanothece halophytica CM1).
SEQ ID NO:19: Shows the DNA sequence coding for the NADH dependent amino acid dehydrogenase of Aphanothece halophytica CM1 optimized for the codon usage of C. glutamicum. The sequence is expanded with a 5′-UTR consisting of a BsaI restriction site, a homologous region for assembly cloning, the strong Pg3N3 promoter and a ribosomal binding site. Additionally, the 3′-end is expanded with a random spacer sequence, a homologous region for assembly cloning and a BsaI restriction site.

### A) MATERIALS AND METHODS

### Chemicals

Kanamycin solution from Streptomyces kanamyceticus was purchased from Sigma Aldrich (St. Louis, USA, Cat. no. K0254). If not stated otherwise, all other chemicals were purchased analytically pure from Merck (Darmstadt, Germany), Sigma Aldrich (St. Louis, USA) or Carl-Roth (Karlsruhe, Germany).

### Bacterial Strain

Corynebacterium glutamicum ATCC13032 (Kinoshita S, Udaka S, Shimono M., J. Gen. Appl. Microbiol. 1957; 3(3): 193-205), the Corynebacterium glutamicum Type Strain/Wildtype, is commercially available at the American Type Culture Collection (ATCC) or at the DSMZ-German Collection of Microorganisms and Cell Cultures GmbH under the deposit no. DSM 20300.

### Cultivation for Cell Proliferation

If not stated otherwise, cultivation/incubation procedures were performed as follows herewith:
a. LB broth (MILLER) from Merck (Darmstadt, Germany; Cat. no. 110285) was used to cultivate E. coli strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
b. LB agar (MILLER) from Merck (Darmstadt, Germany, Cat. no. 110283) was used for cultivation of E. coli strains on agar plates. The agar plates were incubated at 30° C. in an INCU-Line® mini incubator from VWR (Radnor, USA).
c. Brain heart infusion broth (BHI) from Merck (Darmstadt, Germany, Cat. no. 110493) was used to cultivate C. glutamicum strains in liquid medium. The liquid cultures (10 ml liquid medium per 100 ml Erlenmeyer flask with 3 baffles) were incubated in the Infors HT Multitron standard incubator shaker from Infors GmbH (Bottmingen, Switzerland) at 30° C. and 200 rpm.
d. Brain heart agar (BHI-agar) from Merck (Darmstadt, Germany, Cat. no. 113825) was used for cultivation of C. glutamicum strains on agar plates. The agar plates were incubated at 30° C. in an incubator from Heraeus Instruments with Kelvitron® temperature controller (Hanau, Germany).
e. For cultivating C. glutamicum after electroporation, BHI-agar (Merck, Darmstadt, Germany, Cat. no. 113825) was supplemented with 134 g/l sorbitol (Carl Roth GmbH+Co. KG, Karlsruhe, Germany), 2.5 g/l yeast extract (Oxoid/ThermoFisher Scientific, Waltham, USA, Cat. no. LP0021) and 25 mg/l kanamycin. The agar plates were incubated at 30° C. in an incubator from Heraeus Instruments with Kelvitron® temperature controller (Hanau, Germany).

### Determining Optical Density of Bacterial Suspensions

a. The optical density of bacterial suspensions in shake flask cultures was determined at 600 nm (OD600) using the Bio-Photometer from Eppendorf AG (Hamburg, Germany).
b. The optical density of bacterial suspensions produced in the Wouter Duetz (WDS) micro fermentation system (24-Well Plates) was determined at 660 nm (OD660) with the GENios™ plate reader from Tecan Group AG (Männedorf, Switzerland).

### Centrifugation

a. Bacterial suspensions with a maximum volume of 2 ml were centrifuged in 1.5 ml or 2 ml reaction tubes (e.g. Eppendorf Tubes®®3810X) using an Eppendorf 5417 R benchtop centrifuge (5 min. at 13.000 rpm).
b. Bacterial suspensions with a maximum volume of 50 ml were centrifuged in 15 ml or 50 ml centrifuge tubes (e.g. Falcon™ 50 ml Conical Centrifuge Tubes) using an Eppendorf 5810 R benchtop centrifuge for 10 min. at 4.000 rpm.

### DNA Isolation

Plasmid DNA was isolated from E. coli cells using the QIAprep Spin Miniprep Kit from Qiagen (Hilden, Germany, Cat. No. 27106) according to the instructions of the manufacturer.

### Polymerase Chain Reaction (PCR)

PCR with a proof reading (high fidelity) polymerase was used to amplify a desired segment of DNA for sequencing or DNA assembly. Non-proof-reading polymerase Kits were used for determining the presence or absence of a desired DNA fragment directly from E. coli or C. glutamicum colonies.
a. The Phusion® High-Fidelity DNA Polymerase Kit (Phusion Kit) from New England BioLabs Inc. (Ipswich, USA, Cat. No. M0530) was used for template-correct amplification of selected DNA regions according to the instructions of the manufacturer (see Table 2).

TABLE 2
Thermocycling conditions for PCR with Phusion ® High-Fidelity
DNA Polymerase Kit from New England BioLabs Inc.
PCR Program

| | Time | T | |
|---|---|---|---|
| Step | [min.:sec.] | [° C.] | Description |
| 1 | 00:30 | 98 | Initial denaturation step |
| 2 | 00:05 | 98 | Denaturation step |
| 3 | 00:30 | 60 | Annealing step |
| 4 | 30 sec. per kb DNA | 72 | Elongation step |
| | | | Repeat step 2 to 4: 35 x |
| 5 | 05:00 | 72 | Final elongation step |
| 6 | Hold | 4 | Cooling step |

b. Taq PCR Core Kit (Taq Kit) from Qiagen (Hilden, Germany, Cat. No. 201203) was used to amplify a desired segment of DNA to confirm its presence. The kit was used according to the instructions of the manufacturer (see Table 3).

TABLE 3
Thermocycling conditions for PCR with
Taq PCR Core Kit (Taq Kit) from Qiagen.
PCR Program

| | Time | T | |
|---|---|---|---|
| Step | [min.:sec.] | [° C.] | Description |
| 1 | 05:00 | 94 | Initial denaturation step |
| 2 | 00:30 | 94 | Denaturation step |
| 3 | 00:30 | 52 | Annealing step |
| 4 | 1 min. per kb DNA | 72 | Elongation step |
| | | | Repeat step 2 to 4: 35 x |
| 5 | 04:00 | 72 | Final elongation step |
| 6 | Hold | 4 | Cooling step |

c. SapphireAmp® Fast PCR Master Mix (Sapphire Mix) from Takara Bio Inc (Takara Bio Europe S.A.S., Saint-Germain-en-Laye, France, Cat. No. RR350A/B) was used as an alternative to confirm the presence of a desired segment of DNA in cells taken from E. coli or C. glutamicum colonies according to the instructions of the manufacturer (see Table 4).

TABLE 4
Thermocycling conditions for PCR with SapphireAmp ®
Fast PCR Master Mix (Sapphire Mix) from Takara Bio Inc.
PCR Program

| | Time | T | |
|---|---|---|---|
| Step | [min.:sec.] | [° C.] | Description |
| 1 | 01:00 | 94 | Initial denaturation step |
| 2 | 00:05 | 98 | Denaturation step |
| 3 | 00:05 | 55 | Annealing step |
| 4 | 10 sec. per kb DNA | 72 | Elongation step |
| | | | Repeat step 2 to 4: 30 x |
| 5 | 04:00 | 72 | Final elongation step |
| 6 | Hold | 4 | Cooling step |

d. All oligonucleotide primers were synthesized by Eurofins Genomics GmbH (Ebersberg, Germany).
e. As PCR template either a suitably diluted solution of isolated plasmid DNA or of total DNA isolated from a liquid culture or the total DNA contained in a bacterial colony (colony PCR) was used. For said colony PCR the template was prepared by taking cell material with a sterile toothpick from a colony on an agar plate and placing the cell material directly into the PCR reaction tube. The cell material was heated for 10 sec. with 800 W in a microwave oven type Mikrowave & Grill from SEVERIN Elektrogeräte GmbH (Sundern, Germany) and then the PCR reagents were added to the template in the PCR reaction tube.
f. All PCR reactions were carried out in PCR cyclers type Mastercycler or Mastercycler nexus gradient from Eppendorf AG (Hamburg, Germany).

### Restriction Enzyme Digestion of DNA

For restriction enzyme digestions either “FastDigest restriction endonucleases (FD)” (ThermoFisher Scientific, Waltham, USA) or restriction endonucleases from New England BioLabs Inc. (Ipswich, USA) were used. The reactions were carried out according to the instructions of the manufacturer's manual.

### Determining the Sizes of DNA Fragments

a. The sizes of small DNA fragments (<1000 bps) were usually determined by automatic capillary electrophoresis using the QIAxcel from Qiagen (Hilden, Germany).
b. If DNA fragments needed to be isolated or if the DNA fragments were >1000 bps DNA was separated by TAE agarose gel electrophoresis and stained with GelRed® Nucleic Acid Gel Stain (Biotium, Inc., Fremont, Canada). Stained DNA was visualized at 302 nm.

### Purification of PCR Amplificates and Restriction Fragments

PCR amplificates and restriction fragments were cleaned up using the QIAquick PCR Purification Kit from Qiagen (Hilden, Germany; Cat. No. 28106), according to the manufacturer's instructions. DNA was eluted with 30 μl 10 mM Tris*HCl (pH 8.5).

### Determining DNA Concentration

DNA concentration was measured using the NanoDrop Spectrophotometer ND-1000 from PEQLAB Biotechnologie GmbH, since 2015 VWR brand (Erlangen, Germany).

### Assembly Cloning

Plasmid vectors were assembled using the “NEBuilder HiFi DNA Assembly Cloning Kit” purchased from New England BioLabs Inc. (Ipswich, USA, Cat. No. E5520). The reaction mix, containing the linear vector and at least one DNA insert, was incubated at 50° C. for 60 min. 0.5 μl of Assembly mixture was used for each transformation experiment.

Chemical Transformation of E. coli

For plasmid cloning, chemically competent “NEBO Stable Competent E. coli (High Efficiency)” (New England BioLabs Inc., Ipswich, USA, Cat. No. C3040) were transformed according to the manufacturer's protocol. Successfully transformed cells were selected on LB agar supplemented with 25 mg/l kanamycin.

Transformation of C. glutamicum

Transformation of C. glutamicum with plasmid-DNA was conducted via electroporation using a “Gene Pulser Xcell” (Bio-Rad Laboratories GmbH, Feldkirchen, Germany) as described by Ruan et al. (2015). Electroporation was performed in 1 mm electroporation cuvettes (Bio-Rad Laboratories GmbH, Feldkirchen, Germany) at 1.8 kV and a fixed time constant set to 5 ms. Transformed cells were selected on BHI-agar containing 134 g/l sorbitol, 2.5 g/l Yeast Extract and 25 mg/l kanamycin.

### Determining Nucleotide Sequences

Nucleotide sequences of DNA molecules were determined by Eurofins Genomics GmbH (Ebersberg, Germany) by cycle sequencing, using the dideoxy chain termination method of Sanger et al. (Proceedings of the National Academy of Sciences USA 74, 5463-5467, 1977). Clonemanager Professional 9 software from Scientific & Educational Software (Denver, USA) was used to visualize and evaluate the sequences as well as for in silico assembly of sequences.

Glycerol Stocks of E. coli and C. glutamicum Strains

For long time storage of E. coli- and C. glutamicum strains glycerol stocks were prepared. Selected E. coli clones were cultivated in 10 ml LB medium supplemented with 2 g/l glucose. Selected C. glutamicum clones were cultivated in 10 ml twofold concentrated BHI medium supplemented with 2 g/l glucose. Media for growing plasmid containing E. coli- and C. glutamicum strains were supplemented with 25 mg/l kanamycin. The medium was contained in 100 ml Erlenmeyer flasks with 3 baffles. It was inoculated with a loop of cells taken from a colony. The culture was then incubated for 18 h at 30° C. and 200 rpm. After said incubation period 1.2 ml 85% (v/v) sterile glycerol were added to the culture. The obtained glycerol containing cell suspension was then aliquoted in 2 ml portions and stored at −80° C.

### GAA Production in Small-Scale Cultivations

The millilitre-scale cultivation system according to Duetz (2007) was used to assess the GAA-production of the strains. For this purpose, 24-deepwell microplates (24 well WDS plates) from EnzyScreen BV (Heemstede, Netherlands, Cat. no. CR1424) filled with 2.5 ml medium per well were used.

Precultures of the strains were done in 10 ml seed medium (SM). The medium was contained in a 100 ml Erlenmeyer flask with 3 baffles. It was inoculated with 100 μl of a glycerol stock culture and the culture was incubated for 24 h at 30° C. and 200 rpm. The composition of the seed medium (SM) is shown in Table 5.

TABLE 5
Seed medium (SM)

| Components | Concentration (g/l) |
|---|---|
| Yeast extract FM902 (Angel Yeast Co., | 10 |
| LTD, Hubei, P.R. China) | |
| Urea | 1.5 |
| KH₂PO₄ | 0.5 |
| K₂HPO₄ | 0.5 |
| MgSO₄ * 7 H₂O | 1 |
| Biotin | 0.0001 |
| Thiamine hydrochloride | 0.0001 |
| FeSO₄ * 7 H₂O | 0.01 |
| MnSO₄ * H₂O | 0.01 |
| Glucose | 20 |
| Kanamycin | 0.025 |
| pH = 7.0 | |

After said incubation period the optical densities OD600 of the precultures were determined. The volume, needed to inoculate 2.5 ml of production medium (PM) to an OD600 of 0.1, was sampled from the preculture, centrifuged (1 mc at 8000 g) and the supernatant was discarded. Cells were then resuspended in 100 μl of production medium.

The main cultures were started by inoculating the 2.4 ml production medium (PM) containing wells of the 24 Well WDS-Plate with each 100 μl of the resuspended cells from the precultures. The composition of the production medium (PM) is shown in Table 6.

TABLE 6
Production medium (PM)

| Components | Concentration (g/l) |
|---|---|
| 3-(N-morpholino)propanesulfonic acid (MOPS) | 40 |
| Yeast extract FM902 (Angel Yeast Co., | 1.5 |
| LTD, Hubei, P.R. China) | |
| (NH₄)₂SO₄ | 10 |
| NH₄Cl | 15 |
| Trisodium citrate * 2 H₂O | 10 |
| Urea | 1 |
| KH₂PO₄ | 0.5 |
| K₂HPO₄ | 0.5 |
| Ammonium acetate | 7.7 |
| MgSO₄ * 7 H₂O | 1 |
| Biotin | 0.0001 |
| Thiamine hydrochloride | 0.0001 |
| FeSO₄ * 7 H₂O | 0.01 |
| MnSO₄ * H₂O | 0.01 |
| ZnSO₄ * 7 H₂O | 0.00002 |
| CuSO₄ * 5 H₂O | 0.0004 |
| Antifoam XFO-1501 (Ivanhoe | 0.5 |
| Industries Inc., Zion, USA) | |
| Glucose | 40 |
| L-Arginine | 1.9 |
| Kanamycin | 0.025 |
| pH = 7.2 | |

The main cultures were incubated for 72 h at 30° C. and 225 rpm in an Infors HT Multitron standard incubator shaker from Infors GmbH (Bottmingen, Switzerland) until complete consumption of glucose. The glucose concentration in the suspension was analyzed with the blood glucose-meter OneTouch Vita® from LifeScan (Johnson & Johnson Medical GmbH, Neuss, Germany).

After cultivation the culture suspensions were transferred to a deep well microplate. A part of the culture suspension was suitably diluted to measure the OD600. Another part of the culture was centrifuged and the concentration of GAA in the supernatant was analyzed as described below.

### Quantification of GAA

Samples were analyzed with an analyzing system from Agilent, consisting of a HPLC “Infinity 1260” coupled with a mass analyzer “Triple Quad 6420” (Agilent Technologies Inc., Santa Clara, USA). Chromatographic separation was done on the Atlantis HILIC Silica column, 4.6×250 mm, 5 μm (Waters Corporation, Milford, USA) at 35° C. Mobile phase A was water with 10 mM ammonium formate and 0.2% formic acid. Mobile phase B was a mixture of 90% acetonitrile and 10% water, 10 mM ammonium formate were added to the mixture. The HPLC system was started with 100% B, followed by a linear gradient for 22 min and a constant flow rate of 0.6 mL/min to 66% B. The mass analyzer was operated in the ESI positive ionization mode. For detection of GAA the m/z values were monitored by using an MRM fragmentation [M+H]+ 118-76. The limit of quantification (LOQ) for GAA was fixed to 7 ppm.

### B) EXPERIMENTAL RESULTS

### Example 1: Cloning of the Gene AGAT-Mp Coding for an L-Arginine:Glycine Amidinotransferase (AGAT, EC 2.1.4.1) from Moorea producens

Moorea producens is a filamentous cyanobacterium. The genome of the Moorea producens strain PAL-8-15-08-1 was published by Leao et al. (Leao T, Castelão G, Korobeynikov A, Monroe E A, Podell S, Glukhov E, Allen E E, Gerwick W H, Gerwick L, Proc Natl Acad Sci USA. 2017 Mar. 21; 114(12):3198-3203. doi: 10.1073/pnas.1618556114; Genbank accession Number CP017599.1). It contains an open reading frame coding for a L-arginine:glycine amidinotransferase (AGAT, EC 2.1.4.1; locus_tag BJP34_00300 shown in SEQ ID NO: 1). SEQ ID NO: 2 shows the derived amino acid sequence (Genbank accession Number WP_070390602).

Using the software tool “Optimizer” (http://genomes.urv.es/OPTIMIZER/) the amino acid sequence was translated back into a DNA sequence optimized for the codon usage of C. glutamicum. The 5′-end of the optimized gene was expanded with a BsaI restriction site, a 5′-UTR sequence for assembly cloning and a ribosomal binding site. At the 3-end a second stop-codon, a sequence for assembly cloning and a BsaI-site was added. The resulting DNA sequence (SEQ ID NO: 3) was ordered for gene synthesis from Eurofins Genomics GmbH (Ebersberg, Germany) and it was delivered as part of a cloning plasmid with an ampicillin resistance gene (designated as pEX-A258_AGAT-Mp).

The E. coli-C. glutamicum shuttle plasmid pLIB_P consists of the replication origin from pBL1 (for C. glutamicum), the pSC101 replication origin (for E. coli) and a kanamycin resistance gene. Following a unique NotI restriction site it has a strong promoter, two inversely orientated BsaI-sites and the BioBricks Terminator BBa_B1006 (SEQ ID NO: 5).

pLIB_P was digested using the restriction endonuclease BsaI and the DNA was purified with the “QIAquick PCR Purification Kit” (Qiagen GmbH, Hilden, Germany).

The cloning plasmid pEX-A258_AGAT-Mp was digested using the restriction endonuclease BsaI and the DNA was purified with the “QIAquick PCR Purification Kit” (Qiagen GmbH, Hilden, Germany).

The DNA solutions of BsaI digested pLIB_P and pEX-A258_AGAT-Mp were joined, and matching sequence ends were assembled using the “NEBuilder HiFi DNA Assembly Cloning Kit” (New England BioLabs Inc., Ipswich, USA, Cat. No. E5520). The product was transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmid was named pLIB_P_AGAT-Mp.

### Example 2: Synthesis of a Gene Coding for an NADH Dependent AaDH from Mycobacterium tuberculosis H37Ra

The open reading frame MRA_2804 of Mycobacterium tuberculosis H37Ra presumably codes for an NADH dependent amino acid dehydrogenase (Genbank accession CP000611 locus_tag=“MRA_2804”, SEQ ID NO:7). SEQ ID NO: 8 shows the derived amino acid sequence.

Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a BsaI restriction site, a homologous region for assembly cloning, the strong Pg3N3 promoter and a ribosomal binding site. Additionally, the 3′-end was expanded with a random spacer sequence, a homologous region for assembly cloning and a BsaI restriction site. The resulting DNA sequence (SEQ ID NO: 9) was ordered for gene synthesis from Invitrogen/Geneart (Thermo Fisher Scientific, Waltham, USA) and it was delivered as part of a cloning plasmid with an ampicillin resistance gene.

### Example 3: Synthesis of a Gene Coding for an NADH Dependent AaDH from Mycobacterium smegmatis MC2 155

The open reading frame LJ00_13235 of Mycobacterium smegmatis MC2 155 presumably codes for an NADH dependent amino acid dehydrogenase (Genbank accession CP009494 locus_tag=“LJ00_13235”, SEQ ID NO:11). SEQ ID NO:12 shows the derived amino acid sequence.

Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a BsaI restriction site, a homologous region for assembly cloning, the strong Pg3N3 promoter and a ribosomal binding site. Additionally, the 3′-end was expanded with a random spacer sequence, a homologous region for assembly cloning and a BsaI restriction site. The resulting DNA sequence (SEQ ID NO: 13) was ordered for gene synthesis from Invitrogen/Geneart (Thermo Fisher Scientific, Waltham, USA) and it was delivered as part of a cloning plasmid with an ampicillin resistance gene.

### Example 4: Synthesis of a Gene Coding for an NADH Dependent AaDH from Bacillus subtillis 168

The open reading frame HIR77_18035 of Bacillus subtilis 168 presumably codes for an NADH dependent amino acid dehydrogenase (Genbank accession CP053102 locus_tag=“HIR77_18035”, SEQ ID NO:15). SEQ ID NO:16 shows the derived amino acid sequence. Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a BsaI restriction site, a homologous region for assembly cloning, the strong Pg3N3 promoter and a ribosomal binding site. Additionally, the 3′-end was expanded with a random spacer sequence, a homologous region for assembly cloning and a BsaI restriction site. The resulting DNA sequence (SEQ ID NO: 17) was ordered for gene synthesis from Invitrogen/Geneart (Thermo Fisher Scientific, Waltham, USA) and it was delivered as part of a cloning plasmid with an ampicillin resistance gene.

### Example 5: Synthesis of a Gene Coding for an NADH Dependent AaDH from Streptomyces fradiae ATCC10745

The open reading frame CP974_05185 of Streptomyces fradiae ATCC10745 presumably codes for an NADH dependent amino acid dehydrogenase (Genbank accession CP023696 locus_tag=“CP974_05185”, SEQ ID NO:19). SEQ ID NO:20 shows the derived amino acid sequence.

Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a BsaI restriction site, a homologous region for assembly cloning, the strong Pg3N3 promoter and a ribosomal binding site. Additionally, the 3′-end was expanded with a random spacer sequence, a homologous region for assembly cloning and a BsaI restriction site. The resulting DNA sequence (SEQ ID NO:21) was ordered for gene synthesis from Invitrogen/Geneart (Thermo Fisher Scientific, Waltham, USA) and it was delivered as part of a cloning plasmid with an ampicillin resistance gene.

### Example 6: Synthesis of a Gene Coding for an NADH Dependent AaDH from Aphanothece halophytica CM1

Aphanothece halophytica CM1 has an open reading frame presumably coding for an NADH dependent amino acid dehydrogenase (Genbank accession MG430510, SEQ ID NO:23). SEQ ID NO:25 shows the derived amino acid sequence.

Using the software tool “Codon Optimization Tool” (Integrated DNA Technologies Inc., Coralville, Iowa, USA) the open reading frame was optimized for the codon usage of C. glutamicum. The resulting sequence was expanded with a 5′-UTR consisting of a BsaI restriction site, a homologous region for assembly cloning, the strong Pg3N3 promoter and a ribosomal binding site. Additionally, the 3′-end was expanded with a random spacer sequence, a homologous region for assembly cloning and a BsaI restriction site. The resulting DNA sequence (SEQ ID NO:24) was ordered for gene synthesis from Invitrogen/Geneart (Thermo Fisher Scientific, Waltham, USA) and it was delivered as part of a cloning plasmid with an ampicillin resistance gene.

### Example 7: Cloning of Plasmids for the Co-Expression of Amino Acid Dehydrogenases and AGAT-Mp

To enable the combined expression of AGAT-Mp and each amino acid dehydrogenase, the dehydrogenase genes were cloned into plasmid pLIB_P_AGAT-Mp.

The Plasmid pLIB_P_AGAT-Mp digested using the restriction endonuclease NotI and the DNA was purified with the “QIAquick PCR Purification Kit” (Qiagen GmbH, Hilden, Germany).

Each of the five plasmids containing the synthetic amino acid dehydrogenase genes was digested using the restriction endonuclease BsaI and the resulting DNAs were purified with the “QIAquick PCR Purification Kit” (Qiagen GmbH, Hilden, Germany).

The DNA of NotI digested pLIB_P_AGAT-Mp was joined with each of the BsaI digested amino acid dehydrogenase genes and the matching sequence ends were assembled using the “NEBuilder HiFi DNA Assembly Cloning Kit” (New England BioLabs Inc., Ipswich, USA, Cat. No. E5520).

The products were transformed into “NEB Stable Competent E. coli (High Efficiency)” (New England Biolabs, Ipswich, USA) and cells were grown on LB agar containing 25 mg/l kanamycin. Proper plasmid clones were identified by restriction digestion and DNA sequencing. The resulting plasmids are shown in Table 7.

TABLE 7
Plasmids for the co-expression of amino
acid dehydrogenases and AGAT-Mp

| Plasmid | AaDH gene |
|---|---|
| pLIB_AaDH-Mt_AGAT-Mp | AaDH from |
| | Mycobacterium tuberculosis H37Ra |
| pLIB_AaDH-Ms_AGAT-Mp | AaDH from |
| | Mycobacterium smegmatis MC2 155 |
| pLIB_AaDH-Bs_AGAT-Mp | AaDH from |
| | Bacillus subtilis 168 |
| pLIB_AaDH-Sf_AGAT-Mp | AaDH from |
| | Streptomyces fradiae ATCC10745 |
| pLIB_AaDH-Ah_AGAT-Mp | AaDH from |
| | Aphanothece halophytica CM1 |

### Example 8: Transformation of C. glutamicum ATCC13032 with the Expression Plasmids

Corynebacterium glutamicum ATCC13032 (Kinoshita et al., J. Gen. Appl. Microbiol. 1957; 3(3): 193-205) was transformed with the expression plasmids by electroporation and plasmid containing cells were selected with 25 mg/l kanamycin. The resulting plasmid containing strains are shown in Table 8.

TABLE 8
Plasmid containing derivatives of C. glutamicum ATCC13032

| Recipient strain | Plasmid | Resulting strain |
|---|---|---|
| ATCC13032 | pLIB_P_AGAT-Mp | ATCC13032/pLIB_P_AGAT-Mp |
| ATCC13032 | pLIB_AaDH-Mt_AGAT-Mp | ATCC13032/pLIB_AaDH-Mt_AGAT-Mp |
| ATCC13032 | pLIB_AaDH-Ms_AGAT-Mp | ATCC13032/pLIB_AaDH-Ms_AGAT-Mp |
| ATCC13032 | pLIB_AaDH-Bs_AGAT-Mp | ATCC13032/pLIB_AaDH-Bs_AGAT-Mp |
| ATCC13032 | pLIB_AaDH-Sf_AGAT-Mp | ATCC13032/pLIB_AaDH-Sf_AGAT-Mp |
| ATCC13032 | pLIB_AaDH-Ah_AGAT-Mp | ATCC13032/pLIB_AaDH-Ah_AGAT-Mp |

### Example 9: Impact of AaDH Activity on GAA Production

To assess the impact of the expression of AaDH genes on GAA production, strains ATCC13032/pLIB_P_AGAT-Mp, ATCC13032/pLIB_AaDH-Mt_AGAT-Mp, ATCC13032/pLIB_AaDH-Ms_AGAT-Mp, ATCC13032/pLIB_AaDH-Bs_AGAT-Mp, ATCC13032/pLIB_AaDH-Sf_AGAT-Mp and ATCC13032/pLIB_AaDH-Ah_AGAT-Mp were cultivated in the Wouter Duetz system in production medium and the resulting GAA titers were determined as described above.

TABLE 9
Impact of the expression of NADH dependent
amino acid dehydrogenases on GAA production

| Strain | GAA |
|---|---|
| ATCC13032/pLIB_P_AGAT-Mp | 122 mg/l |
| ATCC13032/pLIB_AaDH-Mt_AGAT-Mp | 414 mg/l |
| ATCC13032/pLIB_AaDH-Ms_AGAT-Mp | 275 mg/l |
| ATCC13032/pLIB_AaDH-Bs_AGAT-Mp | 391 mg/l |
| ATCC13032/pLIB_AaDH-Sf_AGAT-Mp | 202 mg/l |
| ATCC13032/pLIB_AaDH-Ah_AGAT-Mp | 361 mg/l |

The cultivation of strains containing AaDH genes resulted in higher GAA titers, compared to ATCC13032/pLIB_P_AGAT-Mp (see Table 9). We conclude that the expression of genes, coding for NADH dependent amino acid dehydrogenases, improves the production of GAA.

1. A microorganism, comprising:
at least one heterologous gene coding for a protein having a function of a L-arginine:glycine amidinotransferase, and
at least one heterologous gene coding for a protein having a function of a NADH-dependent amino acid dehydrogenase.

2. The microorganism of claim 1, wherein an activity of the protein having the function of a NADH-dependent amino acid dehydrogenase is increased compared with a respective activity in a wildtype microorganism.

3. The microorganism of claim 1, wherein the protein having the function of a NADH-dependent amino acid dehydrogenase is selected from the group consisting of alanine dehydrogenase (EC 1.4.1.1), glycine dehydrogenase (EC 1.4.1.10) and aspartate dehydrogenase (EC 1.4.1.21).

4. The microorganism of claim 1, wherein the microorganism has an increased ability to produce L-arginine from L-ornithine compared with the ability of the wildtype microorganism.

5. The microorganism of claim 4, wherein the microorganism has an increased activity of an enzyme having a function of a carbamoylphosphate synthase compared to the respective enzymic activity in the wildtype microorganism.

6. The microorganism of claim 4, wherein the microorganism further comprises an enzyme having function of an argininosuccinate lyase with an increased activity compared to the respective enzymic activity in the wildtype microorganism.

7. The microorganism of claim 4, wherein the microorganism further comprises an enzyme having a function of an omithine carbamoyltransferase with an increased activity compared to the respective enzymic activity in the wildtype microorganism.

8. The microorganism of claim 4, wherein the microorganism further comprises an enzyme having a function of an argininosuccinate synthetase with an increased activity compared to the respective enzymic activity in the wildtype microorganism.

9. The microorganism of claim 4,
wherein the microorganism comprises at least one enzyme selected from the group consisting of an enzyme having a function of a carbamoylphosphate synthase with an increased activity compared to the respective enzymic activity in the wildtype microorganism, an enzyme having a function of an argininosuccinate lyase with an increased activity compared to the respective enzymic activity in the wildtype microorganism, an enzyme having a function of an omithine carbamoyltransferase with an increased activity compared to the respective enzymic activity in the wildtype microorganism, and an enzyme having a function of an argininosuccinate synthetase with an increased activity compared to the respective enzymic activity in the wildtype microorganism,
wherein increased activity of the at least one enzyme is achieved by overexpressing at least one gene encoding the respective at least one enzyme.

10. The microorganism of claim 1, wherein an expression of a gene encoding a protein having a function of a malate synthase is attenuated compared to an expression of a respective gene in the wildtype microorganism or wherein a gene encoding the protein having the function of a malate synthase is inactivated or deleted.

11. The microorganism of claim 4, wherein an expression of an argR gene coding for Ml arginine responsive repressor protein ArgR is attenuated compared to an expression of the argR gene in the wildtype microorganism or wherein the argR gene is inactivated or deleted.

12. The microorganism of claim 4, wherein at least one or more of the genes coding for an enzyme of a biosynthetic pathway of L-arginine, comprising gdh, argJ, argB, argC and/or argD coding for a glutamate dehydrogenase, an ornithine acetyltransferase, an acetylglutamate kinase, an acetylglutamylphosphate reductase and for an acetylornithine aminotransferase, respectively, is overexpressed.

13. The microorganism of claim 1, wherein the protein having the function of an L-arginine:glycine amidinotransferase comprises an amino acid sequence which is at least 80% identical to an amino acid sequence according to SEQ ID NO: 2.

14. The microorganism of claim 1, wherein the protein having the function of a NADH-dependent amino acid dehydrogenase comprises an amino acid sequence which is at least 80% identical to La amino acid sequence according to SEQ ID NO: 6, according to SEQ ID NO: 9, according to SEQ ID NO: 12, according to SEQ ID NO: 15 or according to SEQ ID NO: 18.

15. The microorganism of claim 1, wherein the microorganism is selected from the group consisting of the genus Corynebacterium, the genus Enterobacteriaceae and the genus Pseudomonas.

16. A method for the fermentative production of guanidino acetic acid (GAA), the method comprising:
a) cultivating the microorganism as defined in claim 1 in a suitable medium under suitable conditions, and
b) accumulating GAA in the suitable medium to form a GAA containing fermentation broth.

17. The method of claim 16, further comprising isolating GAA from the GAA containing fermentation broth.

18. A The microorganism as claimed in claim 1, further comprising a gene coding for an enzyme having an activity of a guanidinoacetate N-methyltransferase.

19. The microorganism of claim 18, wherein the gene coding for an enzyme having an activity of a guanidinoacetate N-methyltransferase is overexpressed.

20. A method for a fermentative production of creatine, the method comprising:
a) cultivating the microorganism as defined in claim 18 in a suitable medium under suitable conditions, and
b) accumulating creatine in the suitable medium to form a creatine containing fermentation broth.

21. The method of claim 20, further comprising isolating creatine from the creatine containing fermentation broth.


SEQUENCE LISTING (19 sequences)

SEQ ID NO: 1 - DNA, 1146 bp, Moorena producens
  source 1..1146: mol_type = genomic DNA; strain = Moorena producens PAL-8-15-08-1
atgtcggaaaaaattgttaattcctggaatgaatgggatgaattggaagaaatggtggtaggaattgcagactatgctagctttgaaccaaaagaaccagggaatcatccgaaattaagaaatcaaaatttagcggaaatcattcctttccccagtggacctaaagaccctaaagtccttgaaaaagctaatgaagaattaaatggactggcttatttattaaaagaccacgatgtgatagtaagaagacccgaaaaaattgattttactaaatctctaaaaacaccttactttgaagttgcaaatcaatactgtggagtctgtcctcgggatgtcatgattacctttgggaatgaaatcatggaagcgactatgtcgaagagagctagattttttgaatacttaccttaccggaaattggtctatgaatattggaataaagacgagcatatgatttggaatgctgcgcctaaaccgactatgcaggatagtatgtatctagagaatttctgggagctgtctttagaagaacgatttaagcgtatgcatgattttgaattttgtattacacaagatgaagtaatttttgatgcggctgactgtagcagattaggaaaggatatattagttcaggaatcgatgacaacaaatagaacaggaattcggtggttaaaaaagcacctagaaccaagaggatttcgggttcaccctgttcattttccccttgattttttcccctcacacattgactgtacgtttgttcctttgcgaccaggtcttattttgacaaaccctgaaagacctatacgggaagaggaggagaagatttttaaagagaatggctgggagttgatcacagttcctcaaccgacttgctcgaatgatgaaatgccaatgttttgccagtccagtaagtggttgtcaatgaatgttctgagtatatcaccgacaaaggttatctgtgaggaaagagaaaaacctctccaagaattgttggataagcatggatttgaggtttttcctttaccctttagacatgtctttgaatttggggggtcttttcattgtgcaacttgggatattcgccgaaaaggtgagtgtgaagattatttaccaaatttaaactatcaaccgatttgtggttaa

SEQ ID NO: 2 - protein, 381 aa, Moorena producens
  source 1..381: note = strain: Moorena producens PAL-8-15-08-1
MSEKIVNSWNEWDELEEMVVGIADYASFEPKEPGNHPKLRNQNLAEIIPFPSGPKDPKVLEKANEELNGLAYLLKDHDVIVRRPEKIDFTKSLKTPYFEVANQYCGVCPRDVMITFGNEIMEATMSKRARFFEYLPYRKLVYEYWNKDEHMIWNAAPKPTMQDSMYLENFWELSLEERFKRMHDFEFCITQDEVIFDAADCSRLGKDILVQESMTTNRTGIRWLKKHLEPRGFRVHPVHFPLDFFPSHIDCTFVPLRPGLILTNPERPIREEEEKIFKENGWELITVPQPTCSNDEMPMFCQSSKWLSMNVLSISPTKVICEEREKPLQELLDKHGFEVFPLPFRHVFEFGGSFHCATWDIRRKGECEDYLPNLNYQPICG

SEQ ID NO: 3 - DNA, 1362 bp, synthetic construct
  source 1..1362: mol_type = other DNA
ggtctccatccgcgtcggagacgaagtaattgacggcagcacctcgggcaaactcgagcgtctgcgggcaagcttcgcataaagacacgacgaattagacaacattagtaatgctggaagaaacaaccgagagcaggaagaacatgtcagaaaagattgttaactcctggaacgaatgggacgaacttgaagaaatggtggttggcatcgcggactacgcttccttcgagccaaaagaaccaggtaaccaccctaagctacgcaaccagaacctggccgagatcatcccattcccatccggcccaaaggacccaaaggttctcgaaaaagcaaacgaagagctgaacggcctcgcatacctcctaaaggaccacgatgttatcgtccgccgcccagaaaaaattgatttcactaagtcccttaaaaccccatacttcgaggtcgctaaccagtactgcggcgtttgcccacgcgacgttatgattaccttcggtaatgagattatggaggctaccatgagcaagcgtgcacgcttcttcgagtacttgccataccgtaagctcgtctacgagtactggaacaaggatgagcatatgatctggaacgctgctccaaagcctacgatgcaggactccatgtacttggagaacttctgggagctgtccctcgaggagcgcttcaagcgcatgcacgacttcgaattttgcattacccaggacgaggttatcttcgacgcagctgactgctcccgcctgggcaaggacatcctggtccaggagtccatgaccaccaaccgcaccggcattcgttggttgaagaagcacctcgaacctcgcggtttccgcgttcacccagtccacttccctctcgacttcttcccttctcacattgactgcactttcgtccctctccgtcccggcctcatccttaccaacccagagcgcccaatccgcgaagaagaagaaaagatcttcaaggagaacggctgggagcttatcaccgtcccacaacctacctgctccaacgacgaaatgcctatgttctgccagtcctcgaagtggctcagcatgaacgtcctttctatctccccaacaaaggttatttgcgaagagcgtgaaaagccactacaggaactcctggacaagcacggcttcgaagtcttcccactcccattccgccacgttttcgaattcggcggctcctttcactgcgccacttgggacatccgccgcaagggcgaatgcgaagactacctcccaaacctgaactaccagcctatctgcggctaataagctcggtacccggggatccgacgtcgcgttaacttaaggtaccgaattctaagcttcacaaaaagagacc

SEQ ID NO: 4 - DNA, 7061 bp, synthetic construct
  source 1..7061: mol_type = other DNA
gacagtaagacgggtaagcctgttgatgataccgctgccttactgggtgcattagccagtctgaatgacctgtcacgggataatccgaagtggtcagactggaaaatcagagggcaggaactgcagaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgccattcgccattcaggctgcctgcagggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatgggcctgcaggcctcagcgcgattcaggcctggtatgagtcagcaacaccttcttcacgaggcagatctcagcgcccccccccccctagcttgtctacgtctgatgctttgaatcggacggacttgccgatcttgtatgcggtgatttttccctcgtttgcccactttttaatggtggccggggtgagagctacgcgggcggcgacctgctgcgctgtgatccaatattcggggtcgttcactggttcccctttctgatttctggcatagaagaacccccgtgaactgtgtggttccgggggttgctgatttttgcgagacttctcgcgcaattccctagcttaggtgaaaacaccatgaaacactagggaaacacccatgaaacacccattagggcagtagggcggcttcttcgtctagggcttgcatttgggcggtgatctggtctttagcgtgtgaaagtgtgtcgtaggtggcgtgctcaatgcactcgaacgtcacgtcatttaccgggtcacggtgggcaaagagaactagtgggttagacattgttttcctcgttgtcggtggtggtgagcttttctagccgctcggtaaacgcggcgatcatgaactcttggaggttttcaccgttctgcatgcctgcgcgcttcatgtcctcacgtagtgccaaaggaacgcgtgcggtgaccacgacgggcttagcctttgcctgcgcttctagtgcttcgatggtggcttgtgcctgcgcttgctgcgcctgtagtgcctgttgagcttcttgtagttgctgttctagctgtgccttggttgccatgctttaagactctagtagctttcctgcgatatgtcatgcgcatgcgtagcaaacattgtcctgcaactcattcattatgtgcagtgctcctgttactagtcgtacatactcatatttacctagtctgcatgcagtgcatgcacatgcagtcatgtcgtgctaatgtgtaaaacatgtacatgcagattgctgggggtgcagggggcggagccaccctgtccatgcggggtgtggggcttgccccgccggtacagacagtgagcaccggggcacctagtcgcggataccccccctaggtatcggacacgtaaccctcccatgtcgatgcaaatctttaacattgagtacgggtaagctggcacgcatagccaagctaggcggccaccaaacaccactaaaaattaatagttcctagacaagacaaacccccgtgcgagctaccaactcatatgcacgggggccacataacccgaaggggtttcaattgacaaccatagcactagctaagacaacgggcacaacacccgcacaaactcgcactgcgcaaccccgcacaacatcgggtctaggtaacactgaaatagaagtgaacacctctaaggaaccgcaggtcaatgagggttctaaggtcactcgcgctagggcgtggcgtaggcaaaacgtcatgtacaagatcaccaatagtaaggctctggcggggtgccataggtggcgcagggacgaagctgttgcggtgtcctggtcgtctaacggtgcttcgcagtttgagggtctgcaaaactctcactctcgctgggggtcacctctggctgaattggaagtcatgggcgaacgccgcattgagctggctattgctactaagaatcacttggcggcgggtggcgcgctcatgatgtttgtgggcactgttcgacacaaccgctcacagtcatttgcgcaggttgaagcgggtattaagactgcgtactcttcgatggtgaaaacatctcagtggaagaaagaacgtgcacggtacggggtggagcacacctatagtgactatgaggtcacagactcttgggcgaacggttggcacttgcaccgcaacatgctgttgttcttggatcgtccactgtctgacgatgaactcaaggcgtttgaggattccatgttttcccgctggtctgctggtgtggttaaggccggtatggacgcgccactgcgtgagcacggggtcaaacttgatcaggtgtctacctggggtggagacgctgcgaaaatggcaacctacctcgctaagggcatgtctcaggaactgactggctccgctactaaaaccgcgtctaaggggtcgtacacgccgtttcagatgttggatatgttggccgatcaaagcgacgccggcgaggatatggacgctgttttggtggctcggtggcgtgagtatgaggttggttctaaaaacctgcgttcgtcctggtcacgtggggctaagcgtgctttgggcattgattacatagacgctgatgtacgtcgtgaaatggaagaagaactgtacaagctcgccggtctggaagcaccggaacgggtcgaatcaacccgcgttgctgttgctttggtgaagcccgatgattggaaactgattcagtctgatttcgcggttaggcagtacgttctagattgcgtggataaggctaaggacgtggccgctgcgcaacgtgtcgctaatgaggtgctggcaagtctgggtgtggattccaccccgtgcatgatcgttatggatgatgtggacttggacgcggttctgcctactcatggggacgctactaagcgtgatctgaatgcggcggtgttcgcgggtaatgagcagactattcttcgcacccactaaaagcggcataaaccccgttcgatattttgtgcgatgaatttatggtcaatgtcgcgggggcaaactatgatgggtcttgttgttgacaatggctgatttcatcaggaatggaactgtcatgctgttatgtgcctggctcctaatcaaagctggggacaatgggttgccccgttgatctgatctagttcggattggcggggcttcactgtatctgggggtggcatcgtgaatagattgcacaccgtagtgggcagtgtgcacaccatagtggccatgagcaccaccacccccagggacgccgacggcgcgaagctctgcgcctggtgcggctcggagatcaagcaatccggcgtcggccggagccgggactactgccgccgctcctgccgccagcgggcgtacgaggcccggcgccagcgcgaggcgatcgtgtccgccgtggcgtcggcagtcgctcgccgagatacgtcacgtgacgaaatgcagcagccttccattccgtcacgtgacgaaactcgggccgcaggtcagagcacggttccgcccgctccggccctgccggacccccggcatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatccgagtccaatgtgtccgtgttcacctcagcggccgcaccggtcggggtaccttgacaaatactttccaatggggtacaatggcgcagtgccatccactccgaggttgataccagcctcctcggtggaatgatcatccgcgtcggagacgaagtaattgacggcagcacctcggggagacctcaaatggtctcgcgttaacttaaggtaccgaattctaagcttcacaaaaaaaaaccccgcccctgacagggcggggttttttttactcagcatgtggatggccagtggtcggaccgagcgctttaattaattagcatggtgacacaagcacagtat

SEQ ID NO: 5 - DNA, 1116 bp, Mycobacterium tuberculosis
  source 1..1116: mol_type = genomic DNA; strain = Mycobacterium tuberculosis H37Ra
atgcgcgtcggtattccgaccgagaccaaaaacaacgaattccgggtggccatcaccccggccggcgtcgcggaactaacccgtcgtggccatgaggtgctcatccaggcaggtgccggagagggctcggctatcaccgacgcggatttcaaggcggcaggcgcgcaactggtcggcaccgccgaccaggtgtgggccgacgctgatttattgctcaaggtcaaagaaccgatagcggcggaatacggccgcctgcgacacgggcagatcttgttcacgttcttgcatttggccgcgtcacgtgcttgcaccgatgcgttgttggattccggcaccacgtcaattgcctacgagaccgtccagaccgccgacggcgcactacccctgcttgccccgatgagcgaagtcgccggtcgactcgccgcccaggttggcgcttaccacctgatgcgaacccaagggggccgcggtgtgctgatgggcggggtgcccggcgtcgaaccggccgacgtcgtggtgatcggcgccggcaccgccggctacaacgcagcccgcatcgccaacggcatgggcgcgaccgttacggttctagacatcaacatcgacaaacttcggcaactcgacgccgagttctgcggccggatccacactcgctactcatcggcctacgagctcgagggtgccgtcaaacgtgccgacctggtgattggggccgtcctggtgccaggcgccaaggcacccaaattagtctcgaattcacttgtcgcgcatatgaaaccaggtgcggtactggtggatatagccatcgaccagggcggctgtttcgaaggctcacgaccgaccacctacgaccacccgacgttcgccgtgcacgacacgctgttttactgcgtggcgaacatgcccgcctcggtgccgaagacgtcgacctacgcgctgaccaacgcgacgatgccgtatgtgctcgagcttgccgaccatggctggcgggcggcgtgccggtcgaatccggcactagccaaaggtctttcgacgcacgaaggggcgttactgtccgaacgggtggccaccgacctgggggtgccgttcaccgagcccgccagcgtgctggcctga

SEQ ID NO: 6 - protein, 371 aa, Mycobacterium tuberculosis
  source 1..371: note = strain: Mycobacterium tuberculosis H37Ra
MRVGIPTETKNNEFRVAITPAGVAELTRRGHEVLIQAGAGEGSAITDADFKAAGAQLVGTADQVWADADLLLKVKEPIAAEYGRLRHGQILFTFLHLAASRACTDALLDSGTTSIAYETVQTADGALPLLAPMSEVAGRLAAQVGAYHLMRTQGGRGVLMGGVPGVEPADVVVIGAGTAGYNAARIANGMGATVTVLDINIDKLRQLDAEFCGRIHTRYSSAYELEGAVKRADLVIGAVLVPGAKAPKLVSNSLVAHMKPGAVLVDIAIDQGGCFEGSRPTTYDHPTFAVHDTLFYCVANMPASVPKTSTYALTNATMPYVLELADHGWRAACRSNPALAKGLSTHEGALLSERVATDLGVPFTEPASVLA

SEQ ID NO: 7 - DNA, 1821 bp, synthetic construct
  source 1..1821: mol_type = other DNA
ttcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatgcgcgtgggtatcccaaccgaaaccaagaacaacgagttccgcgtcgcaatcaccccagctggtgtcgctgagctcacccgccgtggccacgaagtactgatccaggcaggcgccggtgagggctccgccatcaccgatgctgacttcaaagcagccggcgctcaactggtcggcaccgctgaccaggtgtgggccgacgcagacctgctgctgaaggtcaaagaacctattgccgcagaatacggccgcctgcgccacggccagatcctgttcaccttcctccacctggcagcttcccgtgcgtgcaccgacgctctgctggactctggcaccacctcgatcgcttacgagactgtacagactgctgatggtgcactcccacttctcgccccaatgtccgaggtggcaggccgcctcgcagcacaagttggcgcataccacctgatgcgtacccagggcggtcgcggagtcctcatgggcggagtccctggcgtcgaaccagctgacgtcgtagtcatcggagccggcaccgcaggctacaacgccgctcgtatcgcaaacggcatgggtgcaaccgtcaccgtcctggacattaatatcgacaagcttcgccagctcgacgctgaattctgcggtcgtattcacacccgctactcctctgcatatgaacttgagggcgctgtcaagcgtgctgacctggttatcggcgcagtcctggttccaggcgctaaggcaccaaagctcgtgtccaactccctggtcgcacacatgaagcctggcgcagtcctggtcgacatcgctatcgaccaaggcggatgcttcgagggctcccgcccaaccacctacgaccacccaaccttcgccgtgcacgacaccctcttctactgtgttgcaaacatgccagcatccgttccaaagacctccacatacgctctcaccaacgcaaccatgccatacgttctggaactggctgaccacggctggcgcgctgcatgtcgctccaaccctgccctggcgaagggtttgagtacccacgagggtgcactcctgtcagaacgtgtcgcaaccgacttgggcgttcccttcaccgaaccagcctccgtgctcgcttaataagctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgacaaatacgagaccctt

SEQ ID NO: 8 - DNA, 1116 bp, Mycobacterium smegmatis
  source 1..1116: mol_type = genomic DNA; strain = Mycobacterium smegmatis MC2 155
atgctcgtcggaatcccgaccgagatcaagaacaacgagtaccgggtcgccatcaccccggctggtgtggcagagctgacccgcagaggtcacgaggtgatcatccaggccggtgccggtgagggctcggccatctcggatcgcgacttcaaggcggcgggcgccgagatcgtcaacaccgccgaccaggtctggtcggaagcagaactgctgctcaaggtgaaagaaccgatcgagcccgagtactcgcggatgcgcaagggccagaccctgttcacctacctgcacctcgcggcgtccaagccgtgcaccgacgcgctgctggcgtccggcaccacgtcgatcgcctacgagaccgtgcagaccgccgagggcgcgctgcccctgctcgcgccgatgagcgaggtcgccggccggctctcggcccaggtcggcgcctaccacctcatgcgcagctacggtggccgtggcgtcctgatgggcggcgtcccgggtgtcgcgcctgccgaggtcgtcgtgatcggcgcaggcaccgccggctacaacgccgcgcgtgtcgctgcgggcatgggcgcccacgtgacggtgttcgatctcaacatcaacacgttgcgccgcgtcgacggcgagttcggcggacgcatcgagacccgctactcgtcgagcctcgaactcgaagaagcggtcaagaaggccgacctggtgatcggcgcagtcctggtgcccggcgccaaagcgccgaaactggtgaccaattcgactgtcgcgcacatgaagccgggtgcggtgctggtcgacatcgcgatcgaccagggtggctgcttcgaggactcgcggcccaccacgcacgacgagcccacgttcaaggtccacgacacgatcttctactgcgtggccaacatgcccggcgcggtgccgcgcacgtcgacgttcgcgctgaccaactcgaccatgccgtacgtgctcaagctcgccgacaagggctggcaggccgcgtgtgcgtcggattccgcactggccaagggtctctcgacccacgacggcaagctgctgtccgaggccgtcgccaaggatctggacctgccgttcaccgacgcggcacagttcctggcgtaa

SEQ ID NO: 9 - protein, 371 aa, Mycobacterium smegmatis
  source 1..371: note = strain: Mycobacterium smegmatis MC2 155
MLVGIPTEIKNNEYRVAITPAGVAELTRRGHEVIIQAGAGEGSAISDRDFKAAGAEIVNTADQVWSEAELLLKVKEPIEPEYSRMRKGQTLFTYLHLAASKPCTDALLASGTTSIAYETVQTAEGALPLLAPMSEVAGRLSAQVGAYHLMRSYGGRGVLMGGVPGVAPAEVVVIGAGTAGYNAARVAAGMGAHVTVFDLNINTLRRVDGEFGGRIETRYSSSLELEEAVKKADLVIGAVLVPGAKAPKLVTNSTVAHMKPGAVLVDIAIDQGGCFEDSRPTTHDEPTFKVHDTIFYCVANMPGAVPRTSTFALTNSTMPYVLKLADKGWQAACASDSALAKGLSTHDGKLLSEAVAKDLDLPFTDAAQFLA

SEQ ID NO: 10 - DNA, 1821 bp, synthetic construct
  source 1..1821: mol_type = other DNA
ttcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatgctggtcggcatcccaaccgagatcaaaaacaacgaataccgggtcgctatcaccccagcaggtgtggctgagctcacccgccgtggtcacgaggttatcatccaggccggcgcgggcgagggttccgcgatctccgaccgtgatttcaaagcagccggtgctgaaatcgtcaacaccgcagatcaggtctggtcggaagcggagttgctcctgaaggttaaggaaccgatcgaacctgaatactcacgcatgcgcaagggccagaccttgttcacctacctgcacctcgcagcttctaagccatgcactgacgctcttctggcctccggcacaacctccatcgcatacgaaaccgtccagaccgcagaaggagccctccccctcctcgctcctatgtcggaagtcgctggccgcctctccgctcaggttggcgcctaccacctaatgcgctcctacggcggccgtggcgtcctcatgggcggcgttccaggcgttgctccagcagaagttgttgtcatcggcgctggtaccgctggctacaatgcggcacgcgttgcagccggcatgggcgcacatgtcaccgtctttgacctcaacatcaacaccctccgccgcgtcgacggtgagttcggcggccgtatcgaaacccgttactcctccagcctggaactggaggaagcagttaagaaggcggaccttgttattggcgcagtcctggtcccaggtgccaaagccccaaagctcgttaccaactccaccgttgcacacatgaagccaggcgcagtcctcgttgacatcgcaatcgatcagggtggatgcttcgaagactcccgccctaccacccatgacgagcctaccttcaaggtacacgacaccatcttctactgcgtagctaacatgccaggcgcagttccacgaaccagcacctttgctctgactaactccaccatgccatacgtcctgaagctcgcagacaagggctggcaggccgcatgcgcatccgactccgcactcgctaaaggcctctcaacccacgacggaaaactgctgtcagaggctgttgccaaggatctcgacctcccattcactgacgctgcacagttcctcgcttaataagctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgacaaatacgagaccctt

SEQ ID NO: 11 - DNA, 1137 bp, Bacillus subtilis
  source 1..1137: mol_type = genomic DNA; strain = Bacillus subtilis subsp. subtilis str. 168; sub_species = Bacillus subtilis subsp. subtilis
atgatcataggggttcctaaagagataaaaaacaatgaaaaccgtgtcgcattaacacccgggggcgtttctcagctcatttcaaacggccaccgggtgctggttgaaacaggcgcgggccttggaagcggatttgaaaatgaagcctatgagtcagcaggagcggaaatcattgctgatccgaagcaggtctgggacgccgaaatggtcatgaaagtaaaagaaccgctgccggaagaatatgtttattttcgcaaaggacttgtgctgtttacgtaccttcatttagcagctgagcctgagcttgcacaggccttgaaggataaaggagtaactgccatcgcatatgaaacggtcagtgaaggccggacattgcctcttctgacgccaatgtcagaggttgcgggcagaatggcagcgcaaatcggcgctcaattcttagaaaagcctaaaggcggaaaaggcattctgcttgccggggtgcctggcgtttcccgcggaaaagtaacaattatcggaggaggcgttgtcgggacaaacgcggcgaaaatggctgtcggcctcggtgcagatgtgacgatcattgacttaaacgcagaccgcttgcgccagcttgatgacatcttcggccatcagattaaaacgttaatttctaatccggtcaatattgctgatgctgtggcggaagcggatctcctcatttgcgcggtattaattccgggtgctaaagctccgactcttgtcactgaggaaatggtaaaacaaatgaaacccggttcagttattgttgatgtagcgatcgaccaaggcggcatcgtcgaaactgtcgaccatatcacaacacatgatcagccaacatatgaaaaacacggggttgtgcattatgctgtagcgaacatgccaggcgcagtccctcgtacatcaacaatcgccctgactaacgttactgttccatacgcgctgcaaatcgcgaacaaaggggcagtaaaagcgctcgcagacaatacggcactgagagcgggtttaaacaccgcaaacggacacgtgacctatgaagctgtagcaagagatctaggctatgagtatgttcctgccgagaaagctttacaggatgaatcatctgtggcgggtgcttaa

SEQ ID NO: 12 - protein, 378 aa, Bacillus subtilis
  source 1..378: note = subspecies and strain: Bacillus subtilis subsp. subtilis str. 168
MIIGVPKEIKNNENRVALTPGGVSQLISNGHRVLVETGAGLGSGFENEAYESAGAEIIADPKQVWDAEMVMKVKEPLPEEYVYFRKGLVLFTYLHLAAEPELAQALKDKGVTAIAYETVSEGRTLPLLTPMSEVAGRMAAQIGAQFLEKPKGGKGILLAGVPGVSRGKVTIIGGGVVGTNAAKMAVGLGADVTIIDLNADRLRQLDDIFGHQIKTLISNPVNIADAVAEADLLICAVLIPGAKAPTLVTEEMVKQMKPGSVIVDVAIDQGGIVETVDHITTHDQPTYEKHGVVHYAVANMPGAVPRTSTIALTNVTVPYALQIANKGAVKALADNTALRAGLNTANGHVTYEAVARDLGYEYVPAEKALQDESSVAGA

SEQ ID NO: 13 - DNA, 1842 bp, synthetic construct
  source 1..1842: mol_type = other DNA
ttcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatgatcatcggcgtcccaaaggaaatcaagaacaacgagaaccgcgtagccctcaccccaggaggtgtctcccagttaatctccaacggccaccgcgtcctggttgaaaccggcgccggcctcggctccggtttcgaaaacgaggcctacgaatccgcaggcgcggaaatcatcgcagatccaaagcaggtttgggatgctgagatggtcatgaaggttaaggaaccactccctgaagagtacgtctacttccgcaaaggcctcgttctgttcacctacctccacctggcagcagagccagaactggcacaggctttgaaagacaagggcgttaccgcaatcgcgtacgaaaccgtttccgaaggccgcaccctgcctctgctcactcccatgtccgaggtcgcgggacgcatggccgcgcagatcggcgctcaattcctagaaaagccaaaaggtggtaagggcattctactcgcaggcgtaccaggtgtctcccgtggcaaagttactatcatcggcggcggcgtcgtcggtaccaacgcagctaagatggcagttggcctcggagcagacgtcaccatcatcgacttgaacgccgatcgtctccgccagcttgatgacatcttcggccatcaaatcaagacactcatctctaacccagtcaacatcgcagacgcagttgcagaagcagaccttttgatctgcgctgtactcatccctggagcaaaagcacccacgctcgttactgaagagatggttaagcagatgaagccaggctccgtcatcgttgacgttgctatcgatcagggcggtatcgttgaaaccgtcgatcacatcactacccacgaccaaccaacctacgagaaacacggtgtcgttcactacgccgtcgcaaacatgccaggcgcagttccacgtacctccaccattgcactgaccaacgtaactgtaccatacgcacttcagatcgccaacaagggcgctgtgaaggccctggcagataacaccgctctccgtgctggcctgaacaccgctaacggccatgtcacctacgaggcagtcgcacgtgacctgggttacgagtacgttccagcagaaaaagcactccaagacgagtccagcgtcgctggcgcttaataagctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgacaaatacgagaccctt

SEQ ID NO: 14 - DNA, 1116 bp, Streptomyces fradiae
  source 1..1116: mol_type = genomic DNA; strain = Streptomyces fradiae ATCC10745
atgaaggtcggcatcccccgcgaggtcaagaacaacgagttccgcgtcgccatcacccccgccggtgtgcacgagctcgtccgcaacggccatcaggtcttcgtcgagcagaacgccggtgtcggctcctcgatcacggacgacgagtacgtggccgcgggcgcgcgcatcctgcccacggccgacgaggtctgggccaccgcggacctgctcctgaaggtcaaggagcccatcgcggaggagtaccaccgcctccgcaaggaccagaccctcttcacctacctccacctcgccgcctcccgcgagtgcaccgacgccctgctggcctccggcaccaccgcgatcgcgtacgagacggtcgagaccgcgaaccgtgcgctcccgctgctggccccgatgtccgaggtcgccggccgcctcgccccgcaggtcggcgcctaccacctgatgcgcgccaacggcggccgcggcgtcctgccgggcggcgtgcccggcgtggtcccggccaaggccgtggtcatcggcggcggtgtctccggctggaacgccacgcagatcgccgtcggcatgggcttcgacgtcaccctgctcgaccgcgacatcaacaagctgcgcgaggcggacaagatcttcggcacgaaggtcaaggccgtcatgtccaactccttcgagctggagaaggccgtcctcgacgccgacctcgtcatcggcgccgtcctcatcccgggcgccaaggccccgaagctcgtcaccaacgagctggtctcccggatgaagcccggaagtgtccttgtcgacatcgcgatcgaccagggcggctgcttcgaggactcgcgtcccaccacgcacgccgacccgtccttccccgtccacgagtcgatcttctactgcgtggccaacatgccgggcgccgtgccgaacacctccacctacgccctgaccaacgccacgcttccctacatcgtgtcgctggccaacaacggctgggtcgaggcgctccgccgcgacccggcgctcgccaagggcctcaacacgcacgacggcaaggtcgtgtaccggcaggtcgccgaggcgcacggcctcgaccacgtggagctcgcctccctcatcggctga

SEQ ID NO: 15 - protein, 371 aa, Streptomyces fradiae
  source 1..371: note = strain: Streptomyces fradiae ATCC10745
MKVGIPREVKNNEFRVAITPAGVHELVRNGHQVFVEQNAGVGSSITDDEYVAAGARILPTADEVWATADLLLKVKEPIAEEYHRLRKDQTLFTYLHLAASRECTDALLASGTTAIAYETVETANRALPLLAPMSEVAGRLAPQVGAYHLMRANGGRGVLPGGVPGVVPAKAVVIGGGVSGWNATQIAVGMGFDVTLLDRDINKLREADKIFGTKVKAVMSNSFELEKAVLDADLVIGAVLIPGAKAPKLVTNELVSRMKPGSVLVDIAIDQGGCFEDSRPTTHADPSFPVHESIFYCVANMPGAVPNTSTYALTNATLPYIVSLANNGWVEALRRDPALAKGLNTHDGKVVYRQVAEAHGLDHVELASLIG

SEQ ID NO: 16 - DNA, 1821 bp, synthetic construct
  source 1..1821: mol_type = other DNA
ttcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatgaaggtcggcatccctcgcgaggtcaagaacaacgagttccgcgtggcaatcacccctgcaggtgtccacgagctggtccgtaatggccaccaagtctttgtggagcagaacgcaggcgtcggctcctccatcaccgatgatgaatatgttgcagctggcgctcgcatcctgccaaccgcagacgaggtttgggcaactgcagacctgctcctcaaggtcaaggaaccaattgcagaagaataccaccgtctccgcaaggaccaaaccctcttcacctacctgcacctggcggcttcccgcgagtgcactgacgctctcctggcttctggcaccaccgcaatcgcttacgaaaccgttgaaaccgcaaatcgcgcactcccactgctggccccaatgtccgaggttgcaggccgactggccccacaagttggagcttaccacctcatgcgtgctaacggtggccgtggcgtactgccaggcggcgttcctggtgtggttccagctaaggcagtcgtcatcggcggcggtgtgtccggctggaacgcaacccagatcgcagttggcatgggattcgacgttaccctgttggaccgcgatatcaacaagctccgtgaagcagataagatcttcggcaccaaggttaaggctgtcatgtctaactccttcgagctggaaaaggcagtactggatgcagatctggttatcggagctgttcttatcccaggcgctaaggcaccaaagctcgttaccaacgagctggtttcccgaatgaagccaggctccgtcctggttgacatcgctatcgatcagggaggttgcttcgaagactcccgcccaacgactcacgcggatccatcctttcctgttcacgagtccatcttctactgcgttgctaacatgccaggcgctgttccaaacacctctacctacgcgttgaccaacgctaccctaccttacatcgtatctctcgctaacaacggctgggtcgaagcactgcgccgcgatccagctctggcaaaaggcctgaacacccacgatggcaaggttgtgtatcgtcaagtcgctgaagctcacggcctagaccacgtcgaacttgcaagcctcatcggctaataagctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgacaaatacgagaccctt

SEQ ID NO: 17 - DNA, 1083 bp, Aphanothece halophytica
  source 1..1083: mol_type = genomic DNA; strain = Aphanothece halophytica CM1
atggaaatcggcgttcccaaagaaaccaaagaccaagaatatcgggtgggattaagcccgagcagtgttcgcagtttgtgtgagaggaagcatccagtttttgtggaaactcaagcgggagtcggcgcagggtttagtgatcaagactacgaagcagcaggagcaaccgttgttcctgatccaaaatccgcttggaatcgggaactggtagttaaggtgaaagaaccgctccctgatgaatacacttatttacagaaaaatcaagttttatttacttatcttcacttagcagctaatcgtcgtctcaccgaggctttgattgattctggcatcagcgcgatcgcgtatgaaaccgtagaactccccaacgggaaactcccccttctgaacccaatgagtattattgcggggcgtttatcggtacaatttggagcgcgttatctagaaaaacaacaggggggacgcggggttttactcggcggctttcctggggtacgttcaggacgagttgtaattctcggcggtggggttgttgggacggaagctgcacgaatggcggtcggaatcggggcgcaagtccagattttagatgtcaatgtggaacggttagccgaactggaaaacctctttggatcacgagtggaactgctctatagtaactcttcacaaattgaagcggtcgtcccagaagcagatttactgattggcgcagttctgaccacaggaaaacgcgcccccaagttggtgacgcgagaactggtcaaacaaatgcgtgcaggaagtgtgattgttgatgtcgctgtggatcaagggggatgtgtggaaaccttgcaagttacctctcatagtcatcccacttataccgaagaaggagtggttcactttggtgttcctaatatgccaggggctgttccttggacagcaactcaagccttaaacaacagcactttgccttatgtgatccagcttgcggatcaaggcttaaccgccctagaaagcaatccgattttagggaaagggttgaacgttcagaaacagcaattgattcatcctgcggttaaagaagtttttcctgatttatag

SEQ ID NO: 18 - protein, 360 aa, Aphanothece halophytica
  source 1..360: note = strain: Aphanothece halophytica CM1
MEIGVPKETKDQEYRVGLSPSSVRSLCERKHPVFVETQAGVGAGFSDQDYEAAGATVVPDPKSAWNRELVVKVKEPLPDEYTYLQKNQVLFTYLHLAANRRLTEALIDSGISAIAYETVELPNGKLPLLNPMSIIAGRLSVQFGARYLEKQQGGRGVLLGGFPGVRSGRVVILGGGVVGTEAARMAVGIGAQVQILDVNVERLAELENLFGSRVELLYSNSSQIEAVVPEADLLIGAVLTTGKRAPKLVTRELVKQMRAGSVIVDVAVDQGGCVETLQVTSHSHPTYTEEGVVHFGVPNMPGAVPWTATQALNNSTLPYVIQLADQGLTALESNPILGKGLNVQKQQLIHPAVKEVFPDL

SEQ ID NO: 19 - DNA, 1788 bp, synthetic construct
  source 1..1788: mol_type = other DNA
ttcggtctctatccgagtccaatgtgtccgtgttcacctcagcggccattgaagcctaaaaacgaccgagcctattgggattaccattgaagccagtgtgagttgcatcacattggcttcaaatctgagactttaatttgtggattcacgggggtgtaatgtagttcataattaaccccattcgggggagcagatcgtagtgcgaacgatttcaggttcgttccctgcaaaaactatttagcgcaagtgttggaaatgcccccgtttggggtcaatgtccatttttgaatgtgtctgtatgattttgcatctgttgccaaatctttgtttccccgctataattgaggacaggttgacacggagttgactcgacgaattatccaatgtgagtaggtttggtgcgtgagttgaaaaaattcgccatactcgcccttgggttctgtcagctcaagaattcttgagtgaccgatgctctgattgacctaactgcttgacacattgcatttcctacaatcgcgagaggagacacaacatggaaatcggcgtccctaaggaaactaaggaccaggaataccgtgtcggactgagcccatcatccgttcgatccctgtgtgaacgcaagcacccagtcttcgtcgaaacccaggctggcgttggtgcaggcttcagcgatcaggactacgaagcagccggcgcaaccgttgtcccagatccaaagtccgcttggaaccgagaactggttgtcaaggttaaggaaccactcccagatgagtacacctacctgcagaagaaccaggtcctcttcacctacctccacctggcagccaaccgccgcctcaccgaggcactgatcgattccggcatctctgctatcgcgtacgaaaccgtcgagttgcctaacggaaagctcccactgctgaacccaatgtccatcatcgctggccgccttagcgttcaattcggtgcccgctacctggagaagcagcagggcggacgcggtgtcctccttggcggcttcccaggcgttcgctccggccgtgtcgtaatcctgggcggcggcgtcgtcggtaccgaagcagctcgcatggcagttggcatcggtgcacaggtccagatcctggacgtcaacgtcgaacgcctggctgaacttgaaaacctcttcggctcccgcgttgagctcctttactccaactcctcccagatcgaagcagttgtccccgaggctgaccttttgatcggcgctgtacttaccaccggcaagcgcgcaccaaagctggtcacccgtgaacttgtgaagcagatgcgcgcaggatccgttatcgtcgatgtggcagttgaccaaggaggatgcgttgaaacccttcaggttaccagccacagccaccctacctacaccgaagaaggcgtcgtccacttcggcgtgcctaacatgcctggcgcagtcccgtggaccgctacccaggcactgaataactccaccctcccttacgtcatccagctcgcagaccagggcctgaccgctctcgagtcaaatccaatcctcggtaaaggtctgaacgtacagaagcagcaacttatccacccagctgtcaaggaggtcttccctgatctataataagctcggtacccggggatccgacgtcaggaaaggagaggattgatgaattcaggtatctcggagatcgcgagagctcgtaactgcgacggacacctaatgccctttagtaccaaatgggggagttaataaggccgcaccggtcggggtaccttgacaaatacgagaccctt